(19) United States
(12) Patent Application Publication
(10) Pub. No.: US 2018/0328914 A1
(43) Pub. Date: Nov. 15, 2018

(54) Methods for Quantitative Analysis and Targeting of Inflammatory Breast Cancer Tumor Emboli

(71) Applicant: Gayathri Devi, Durham, NC (US)

(72) Inventor: Gayathri Devi, Durham, NC (US)

(21) Appl. No.: 15/972,831

(22) Filed: May 7, 2018

Related U.S. Application Data
(60) Provisional application No. 62/501,868, filed on May 5, 2017.

(51) Int. Cl. G01N 33/50

(52) U.S. Cl. CPC G01N 33/5011

(57) ABSTRACT

The present disclosure provides methods for the quantitative analysis and targeting of inflammatory breast cancer tumor emboli.

### CROSS-REFERENCE TO RELATED APPLICATIONS

This application claims benefit and priority to U.S. Provisional Application 62/501,868 filed May 5, 2017, the contents of which are incorporated by reference in its entirety.

### STATEMENT REGARDING FEDERALLY SPONSORED RESEARCH

This invention was made with government support under RSG-08-290-01 awarded by the National Cancer Institute and W81WXH-13-1-0141 awarded by the Department of Defense. The government has certain rights in the invention.

### BACKGROUND OF THE INVENTION

Inflammatory breast cancer (IBC) is a distinct subtype of advanced breast cancer, which disproportionately affects younger women of childbearing age (Robertson et al. 2010). A critical clinical challenge is that there are very few therapeutic options for IBC patients with metastatic recurrence (Robertson et al. 2010). Even after the use of multimodal treatment strategies (surgery, radiation, chemotherapy), tumor cells in IBC seem to survive, evade cell death, and lead to cancer recurrence (Dressman et al. 2006; Yamauchi et al. 2012). One of the hallmarks of this disease is engorgement of the dermal lymphatics on the chest wall. Morbidities associated with local recurrence include: pain, ulceration, odor, bleeding, lymphedema and the psychological distress of having visible local disease. These changes in the chest wall are due to the presence of clusters of tumor cells that invade skin lymphatics and lymph nodes. It is postulated that the tumor emboli or tumor emboli drive metastasis in this aggressive cancer type (Nguyen et al. 2006; Vermeulen et al. 2010).

In IBC metastasis, tumor cells that separate from the primary tumor mass form multicellular spheroids, termed tumor emboli, which then invade through the lymphatic system and reach distant organs to form secondary tumors. Therefore, it is important to understand the cellular and molecular characteristics of tumor emboli in IBC for better drug development strategies to inhibit metastasis and improve patient survival. Currently, the only preclinical models available to study IBC tumor emboli are: Mary-X, an in vivo triple-negative xenograft model (Alpaugh et al. 1999) and in vitro tumor emboli derived from triple-negative SUM149 and HER-2 overexpressing SUM190 cells (Lehman et al. 2013; Mu et al. 2013). These models have predominantly been used for immunohistochemical analysis and assessment of viability of the tumor emboli as a whole after treatment with anticancer agents. However, current assays do not quantitatively measure cell morphology parameters of the 3D spheroids, both the individual cells that make up the spheroid and the spheroid as a whole.

There is a need to develop a reproducible, high content assay for the comprehensive, quantitative assessment of IBC tumor emboli morphology adapted for high-throughput analysis.

### SUMMARY OF THE INVENTION

In one aspect, the present invention provides a method to simultaneously image multiple cell health characteristics of 3D IBC tumor emboli and quantitatively measure morphological parameters. These methods can be used to determine the effects of cytotoxic compounds on IBC tumor emboli formation and individual tumor cell survival.

In one aspect, the disclosure provides a high-content, high throughput multiparametric assay method for analyzing 3-D Inflammatory Breast Cancer (IBC) tumor emboli comprising: a) generating 3D IBC spheroid array in in vitro culture by (i) plating IBC cells on ultra low attachment plates; (ii) culturing the IBC cells for at least 2 days to form 3-dimensional tumor emboli; b) simultaneously assaying the tumor emboli cell array for at least two cell health parameters selected from the group consisting of cell viability, cell number, nuclear shape, nuclear size, nuclear texture, cell proliferation, and mitochondrial function (mitochondrial membrane potential), wherein measuring comprises simultaneous cell staining, cell imaging, or a combination thereof, and c) analyzing the at least two parameters of step (b). In one particular aspect, step (b) comprises (i) incubating the 3D IBC spheroid array with at least one marker for a cell health parameter selected from the group consisting of Hoechst, YOYO-1 and Mitotracker™ Red.

In another aspect, the disclosure provides a method for high throughput identification of an agent for IBC tumor emboli cell growth inhibition, the method comprising: a) generating a 3D IBC spheroid array in an in vitro culture by (i) plating IBC cells on ultra low attachment plates; and (ii) culturing the IBC cells for at least 2 days to form 3-dimensional tumor emboli; b) providing at least one agent to be tested; c) contacting the IBC tumor emboli array with the agent; and d) detecting at least one signal indicating IBC tumor emboli cells growth inhibition as compared to a non-contacted control.

In yet a further aspect, the disclosure provides an in vitro 3-dimensional Inflammatory Breast Cancer Tumor Emboli high content throughput assay comprising: a) an array of 3-dimensional IBC tumor emboli within multiple culture wells generated in vitro by (i) plating IBC cells in wells of an ultra low attachment plate; (ii) culturing the IBC cells for at least 2 days to form 3-dimensional IBC tumor emboli; b) at least one detection agent able to produce a detectable signal, wherein the detectable signal is selected from the group consisting of (i) a reduction in tumor emboli cell number, (ii) an increase in cell death, (iii) inhibition of spheroid formation, (iv) reduction of mitochondrial integrity, and (v) a combination thereof; c) a microscope for imaging the detectable signal, and d) means for quantitatively analyzing the images.

The foregoing and other aspects and advantages of the invention will appear from the following description. In the description, reference is made to the accompanying drawings which form a part hereof, and in which there are shown, by way of illustration, preferred embodiments of the invention. Such embodiments do not necessarily represent the full scope of the invention, however, and reference is made therefore to the claims and herein for interpreting the scope of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a possible model of how tumor recurrence can develop in IBC when therapy (RT/CT/Targeted) fails to kill all primary tumor cells. In IBC, this involves expansion of residual, apoptosis resistant tumor cells, induction of oxidative stress response, leading to increased TE formation, collective migration, propensity for dermal lymphatic invasion, and metastatic progression.

FIGS. 2A-2F show RTC/TE in vitro Model Flow Chart: A) Using ultra-low attachment plates and specialized media or by B) simulation of lymphatic shear stress and viscosity to mimic lymphatic microenvironment through addition of PEG or hyaluronic acid to normal growth media allow for C) TE formation (Lehaman, 2013). D) This HCA TE system can be modified for a variety of in vitro experiments including invasion, migration as well as expression analysis. E) High-throughput liquid handling allows for rapid, economical screening of multiple compounds in dose-response for F) high content multiparametric analysis. This allows simultaneous measurement of: nuclear morphology (size, aspect ratio, texture—Hoechst), cell proliferation (Hoechst), cell viability (YOYO-1), and mitochondrial function (MitoTracker). For quantitative analysis, an initial threshold for the size of untreated wells is set as indicative of a stable TE and excludes small spheroids. The minimum area cutoff is also set for qualifying mammospheres as 1821 square microns. Fluorescence quantification and localization is determined using a ThermoFisher Celllnsight NXT and 3-channel Cell Health Profiling protocol in HCS Screen software (ThermoFisher). (Unpublished data)

FIG. 3 is a table with characteristics of proposed cell models. (IBC-Inflammatory breast cancer; BC: Breast Cancer; ER: estrogen receptor; PR: progesterone receptor, EGFR/HER2: epidermal growth factor receptors). Currently, we have in hand mCherry-fluorescently labeled and luciferase tagged SUM149, SUM190 and 4T1 cells and HIF-1-GFP reporter lines (Cao, 2013). MCF-7 and HME1 mCherry lines and a stable NFκB reporter SUM149 cell lines are in development. We have also generated unique isotype-matched SUM149 and SUM190 derivatives that are clonal populations of cells selected under chronic oxidative stress stimuli-rSUM149, rSUM190 (Aird, 2012; Aird, 2010; Williams, 2013) that exhibit a tumor recurrent and multi-drug resistance phenotype.

FIGS. 4A-4B show the results of a high content endpoint migration assay. A) Invasion zone quantitation after staining SUM149 treated cells with Hoechst (green) and YOYO-1 (red) dyes. Live (blue) vs. dead (red) cells shown for a single well of a 96 well plate. Well image with stopper in place for 48 h as a no-motility control (left) or removed at 0 h to permit cell migration (right). B) Calculation of % area occupied.

FIG. 5 is a representative image obtained from a transgenic mouse with mammary fat pad window chamber that expresses m-Cherry red fluorescent reporter gene in vascular endothelium, under control of the Tie2 promoter. Tumor cells (blue fluorescence) in this window were labeled with a lipid dye, DiD, prior to transplant.

FIG. 6 is representative images of serial observation of hemoglobin saturation and HIF1 activity during early tumor growth. An increase in arterial pO₂ is the first reaction to the presence of tumor cells (top), followed by increases in venular pO₂ (middle). During the continued process of angiogenesis, HIF-1 expression level increases, suggesting that improvements in pO₂ within the growing tumor mass are not alleviating HIF-1 activity (bottom) (Dewhirst, 2007).

FIG. 7 demonstrates post-RT Recurrent tumor cells show increased tumor invasion and regional migration. Tumor cells migrate along vascular network to unirradiated site. And lead to formation of satellite metastases. The original tumor, with RFP reporter, is at the bottom. Two satellite tumors, in unirradiated tissue, are seen following a track toward the 12 o'clock position. This was not observed in control tumors. Size: 4 mm square. Confocal microscopy image; yellow box outlines area bridging tumor sites; red arrow indicates location of original and satellite tumors. Yellow arrow: shows path of tumor cell migration.

FIG. 8 is a representative figure of lymphatic vasculature visualization in the mammary fat pad within 5-10 min using fluorescently-labeled dextran.

FIGS. 9A-9B demonstrates DSF+Cu effectively inhibits IBC tumor growth through inhibition of NFκB activation. A) In vivo subcutaneous tumor growth studies of IBC PTC with vehicle, DSF alone or DSF+Cu. Arrow shows start of treatment in palpable tumor-bearing mice. B) Western immunoblot analysis of IBC tumors show decreased NFκB and SOD1 antioxidant expression in DSF-Cu treated samples.

FIG. 10 demonstrates MnSOD mimic improves tumor control following RT, while protecting normal tissue. Treatment with MnBuOE pre- and post-RT made tumor xenografts more radiosensitive as indicated by a left shift of the tumor radiation control curve. The TCD50 doses (total RT applied in 5 fractions) were 47Gy (saline controls) and 36.5Gy (MnBuOE), giving a 1.3 dose modifying factor.

FIG. 11 demonstrates DSF-Cu inhibits in vitro tumor emboli formation. SUM149 cells in lymphatic simulating tumor emboli model (Lehman, 2013) treated with DSF, Cu and DSF-Cu at the time of seeding. Spheroids manually counted using phase contrast microscopy on day 4 (N=2, replicates=6).

FIG. 12 is a cartoon representing a model of inflammatory breast cancer development. In IBC, tumor cells that separate from the primary breast tumor mass form multicellular tumor spheroids also referred to as tumor emboli, which then invade through the lymphatic system and reach distant organs to form secondary tumors called metastasis (Inflammatory breast cancer, the disease, the biology and the treatment, CA Cancer Clin J (2010) 60:351-375 and Angiogenesis, lymphangiogenesis, growth pattern, and tumor emboli in inflammatory breast cancer, Cancer (2010) 116(11 Suppl.) 2748-2754).

FIG. 13 depicts the method and results of immunohistochemistry analysis of anti-apoptotic proteins, XIAP and NFκB expression in breast cancer tumor tissue.

FIG. 14 depicts an example of the in vitro high content, high-throughput IBC tumor spheroid assay of the present invention.

FIG. 15 depicts the application of the high content tumor spheroid assay to evaluate efficacy of compounds targeting XIAP and NFκB.

FIG. 16 is a table showing the quantitative data from the images.

FIG. 17 are graphs showing the statistical analysis if image data quantitatively measured by 3 channel cell health profiling protocol (FIG. 16) from 150-174 fields of view per well, 2-3 wells per treatment. One-way ANOVA, Tukey Multiple Comparison Test. Data considered significant if p<0.05.

FIG. 18 is a table summarizing results shown in FIG. 17.

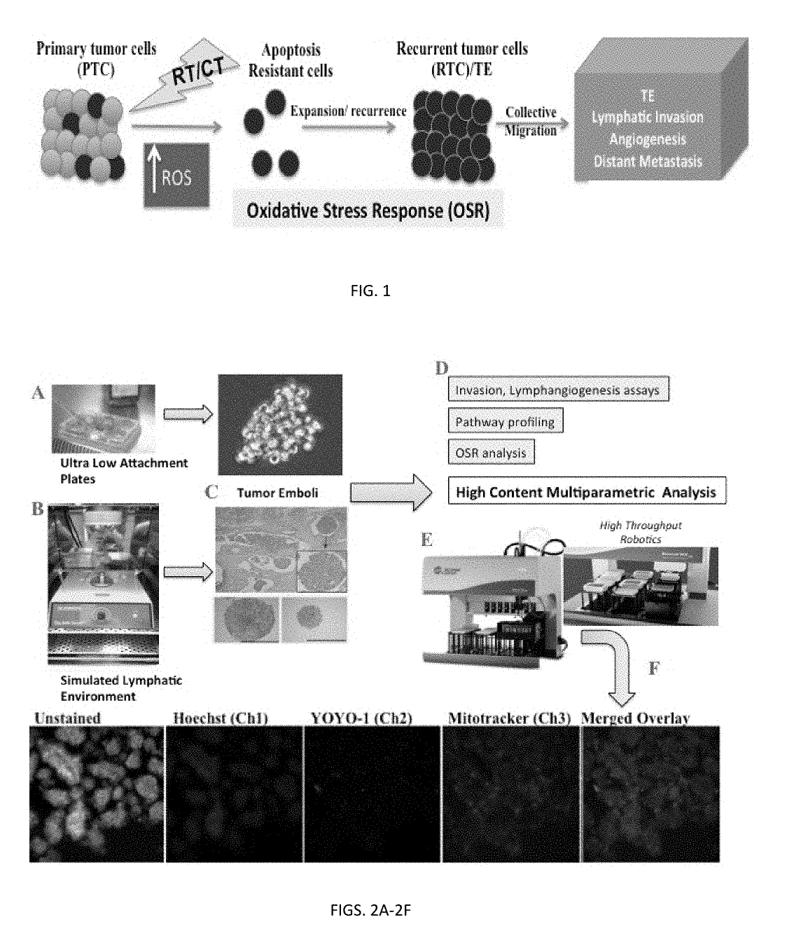

### DETAILED DESCRIPTION OF THE INVENTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

Unless otherwise defined, all technical terms used herein have the same meaning as commonly understood by one of ordinary skill in the art to which this disclosure belongs.

In one embodiment, the present invention provides a method to simultaneously image multiple cell health characteristics of 3D IBC tumor emboli and quantitatively measure morphological parameters. These methods can be used to determine the effects of cytotoxic compounds on IBC tumor emboli formation and individual tumor cell survival.

One embodiment of the present disclosure was to define characteristics of inflammatory breast cancer multicellular tumor emboli and to then use this information to identify therapeutic strategies that can target their formation. Patients with inflammatory breast cancer (IBC) have a very poor prognosis, due to a lack of response to chemotherapy and subsequent metastasis. Previous studies suggest that IBC metastasis is driven by the formation of tumor emboli, which are specialized multicellular structures that collectively migrate and spread to distant organs. It is therefore critical to understand how cells in the tumor emboli evade the process of programmed cell death to develop novel anti-cancer drugs. A high content assay was developed for simultaneous imaging and quantitative analysis of the morphology of tumor emboli growing in 3 dimensional (3D) cultures. Phenotypic readouts include measurement of cell viability, cell number, nuclear shape, nuclear size, nuclear texture, and mitochondrial membrane potential. This 3D IBC tumor emboli high content assay was optimized for use in a high throughput manner and applied to identify novel anti-cancer inhibitors. This assay can be used to for high-throughput drug screening.

In one embodiment, the present invention provides a method to simultaneously imaging multiple cell health characteristics of 3D IBC tumor emboli and quantitatively measure morphological parameters, the method comprising (a) generating an array of 3-dimensional IBC tumor emboli in in vitro culture, (b) in a single reaction mixture, staining the 3D IBC tumor emboli with detectable markers for at least one cell health characteristic; (c) imaging the tumor emboli stained in step (b); and (d) quantitatively measuring at least one morphological parameter of the 3D IBC tumor emboli. In some embodiments, the 3D IBC tumor emboli are incubated with one or more testing agent to screen for testing agents able to inhibit IBC tumor emboli growth or induce IBC tumor emboli cell death. These methods can be used to determine the effects of the tested cytotoxic compounds on IBC tumor emboli formation and individual tumor cell survival.

In another embodiment, the disclosure provides a high-content, high throughput multiparametric assay method for analyzing 3D Inflammatory Breast Cancer (IBC) tumor emboli comprising: a) generating a 3D IBC spheroid array in an in vitro culture by (i) plating IBC cells on ultra low attachment plates; and (ii) culturing the IBC cells for at least 2 days to form 3-dimensional tumor emboli; b) simultaneously assaying the tumor emboli cell array for at least two parameters selected from the group consisting of cell viability, cell number, nuclear shape, nuclear size, nuclear texture, cell proliferation, and mitochondrial function (mitochondrial membrane potential), wherein measuring comprises simultaneous cell staining, cell imaging, or a combination thereof, and c) analyzing the at least two parameters of step (b).

As used herein, cell health characteristics of 3D IBC tumor emboli refers to characteristics or parameters of a cell that inform one skilled in the art of the health of the cell. For example, in suitable embodiments, health cell characteristics (or health cell parameters) include, but are not limited to, cell viability, cell number, nuclear shape, nuclear size, nuclear texture, cell proliferation, and mitochondrial function (mitochondrial membrane potential). The health cell parameters can be generally categorized as cell viability, cell morphology and mitochondrial function. The cell health characteristics include the morphological parameters which are assayed in the methods provided herein. In some embodiments, the stress response like oxidative/reactive oxygen species can be measures. In further embodiments, histological analysis can be performed to determine morphological parameters.

The present methods include a method of generating an array of 3D IBC tumor emboli in in vitro culture for use in the methods described herein, particularly in use in high throughput assays.

3D IBC tumor emboli as used herein are in vitro cultures of IBC tumor cells that form spheroids. IBC tumor emboli are specific and unique heterogeneous cellular structural entities associated with inflammatory breast cancer. IBC tumor emboli are not found in other types of cancer, including breast cancer. IBC tumor emboli are dynamic and show high degree of plasticity as they are composed of more than one cell type, including IBC-associated cancer stem cells, epithelial cells and mesenchymal cells. IBC tumor emboli are heterogeneous in nature, wherein different tumor emboli are composed of different ratios of the three cellular types. IBC tumor emboli instead of a solid mass present as rapidly proliferating diffuse tumor cell clusters. Description of IBC tumor emboli in vitro can be found in Arora, Jay et al. “Inflammatory Breast Cancer Tumor Emboli Express High Levels of Anti-Apoptotic Proteins: Use of a Quantitative High Content and High-Throughput 3D IBC Spheroid Assay to Identify Targeting Strategies.” Oncotarget 8.16 (2017): 25848-25863. PMC. Web. 4 May 2018 and Sauer et al. “Bisphenol A activates EGFR and ERK promoting proliferation, tumor spheroid formation and resistance to EGFR pathway inhibition in estrogen receptor-negative inflammatory breast cancer cells,” Carcinogenesis, Volume 38, Issue 3, 1 Mar. 2017, Pages 252-260, https://doi.org/10.1093/carcin/bgx003, the content of which are incorporated by reference in their entireties.

The term “array” refers to an arrangement of multiple separate areas or regions on a plate or surface on which 3D IBC tumor emboli may be grown, for example, multi-well plates or sections of a culture plate that are distinct from each other. Suitable arrays include, but are not limited to, 6-well, 12-well, 24-well, 48-well, 96-well, 384 well tissue culture plates.

The method of generating 3D IBC tumor emboli encompasses methods of plating IBC cells on ultra-low attachment plates. Suitable ultra-low attachment plates are known in the art and encompass plates which have a suitable coating that inhibits specific and non-specific immobilization, forcing cells into a suspended state and enabling 3D spheroid formation. Suitable plates are available from Corning (Corning, N.Y.) using their Ultra-Low Attachment surface made of hydrophilic, neutrally charged coating covalently bound to a polystyrene vessel surface.

Generating an array of 3D IBC tumor emboli in in vitro culture comprises the steps of plating IBC cells from a single cell suspension onto ultra-low attachment plates in chemically defined medium in which the IBC cells grow and form spheroid tumor emboli. Suitable chemically defined medium include, but are not limited to, for example, Ham's F-12 medium (Invitrogen, Carlsbad, Calif.). In suitable embodiments, the medium is enriched with 5 μg/ml insulin, 1 μg/mL hydrocortisone (Sigma-Aldrich, St. Louis, Mo.), 10 mM HEPES (Invitrogen), 5% FBS (not heat inactivated, Denville Scientific, South Plainfield N.J.). Cells are cultured at known culture conditions, for example, 37° C. under 5% CO₂ conditions.

Suitable IBC cells used for generating the 3D IBC tumor emboli cultures include IBC derived cell lines, for example, but not limited to, SUM149, rSUM149 and SUM190 IBC cells. Other suitable IBC cell lines are contemplated for use in the present invention. The IBC cells are prepared into a single cell suspension by methods known in the art from the 2-D culture, for example, by treatment with trypsin or another enzyme to release the cells from the tissue culture dish.

Additionally, primary IBC cells may be used as the IBC cells in the practice of the present invention. For example, the IBC cells used herein may be primary cells derived from a tissue sample from a patient having IBC. Suitable methods of preparing cells from a tissue sample are known in the art and include, but are not limited to methods of producing single cell suspension s by enzymatic digestion (e.g., trypin, collagenase, dispase, among others and including combinations thereof) of the tissue sample.

The methods described herein provide methods of detecting specific detectable markers within the 3D tumor emboli by methods staining or immunohistochemical staining. Suitable, the methods of staining are done in a one-step process as described in the examples.

In some embodiments, the step of assaying the tumor emboli cell array for at least two parameters selected from the group consisting of cell viability, cell number, nuclear shape, nuclear size, nuclear texture, cell proliferation, and mitochondrial function (mitochondrial membrane potential) encompasses simultaneous cell staining for at least one marker of at least one cell health characteristic, preferably at least two markers of cell health characteristic, more preferably at least three markers of cell health.

Suitable method of evaluating end point parameters are known in the art and include markers, for example, but not limited to, YOYO-1 staining, release of lactate dehydrogenase (LDH) and glutathione (GSH) that occur when cell membranes loses its integrity, propidium iodide and other vital dyes for use in fluorescence microscopy. In a preferred embodiment, the markers use are selected from YOYO-1, Hoescsht, and Mitotracker™ Red. Although other stains are commercially available, these three stains (YOYO-1, Hoescsht, and Mitotracker™ Red) have been optimized to work best in the IBC Tumor Emboli assay described herein.

In one suitable preferred embodiment, the at least one marker selected from the group consisting of Hoechst, YOYO-1 and Mitotracker™ Red. In a preferred embodiment, there are at least three markers, wherein the markers are YOYO-1, Hoescsht, and Mitotracker™ Red.

In some embodiments, measuring the at least one cell health characteristic comprises simultaneous cell staining, cell imaging, or a combination thereof. In a preferred embodiment, both cell staining and cell imaging are performed to measure at least two health cell characteristic parameters, preferably at least three parameters.

Methods of imaging and quantitative analysis of images are known in the art. In some embodiments, the markers are fluorescent markers, and the quantitative analysis comprises comparing the fluorescent signal in a sample compared with a control.

In some embodiments, the method comprises quantitative analysis of images as compared to a control to determine the cell health characteristics of the 3D IBC tumor emboli. Suitable quantitative analysis methods are known in the art, as demonstrated in the Examples provided below.

The control used for comparison is a suitable control for the marker being assayed that is understood by one skilled in the art. For example, in some embodiments, the control may be a sample that is not incubated with the marker (e.g. YOYO-1) in cases in which the level of the marker within a sample is desired. In methods in which the cells are contacted with a cytotoxic test agent and the inhibition of cell growth is being assessed, the control may be a well of cells not treated with the cytotoxic test agent.

In another embodiment, the disclosure provides a method for high throughput identification of an agent for IBC tumor emboli cell growth inhibition, the method comprising: a) generating a 3D IBC spheroid array in an in vitro culture; b) providing at least one agent to be tested; c) contacting the IBC tumor emboli array with the agent; and d) detecting at least one signal indicating IBC tumor emboli cells growth inhibition as compared to a non-contacted control. As described here, a suitable method of generating a 3D IBC spheroid array in an in vitro culture, includes, for example, plating IBC cells on ultra low attachment plates; and culturing the IBC cells for at least 2 days to form 3-dimensional tumor emboli.

The term “agent” or “test agent” or “cytotoxic agent” are used interchangeably herein to refer to a compound or composition being tested for the ability to inhibit IBC tumor emboli cell growth. In some embodiments, the agent may one or more small molecules, peptides, proteins, phage display libraries, antibodies, siRNAs, etc. In one particular embodiment, the agents are agents that inhibit XIAP or NFκB signaling. Suitable anti-XIAP and anti-NFκB agents are known in the art and can be used in the methods described. In other embodiments, the agent may be a panel of untested compounds that is being screened for anti-IBC cancer cell effects.

In some embodiments, a panel of agents with known or unknown properties may be tested in a multi-well format (e.g., 96 well plate) for inhibition of 3D tumor emboli. In other embodiments, a cocktail or mixture or one or more test agents can be used as a composition for the testing.

The term inhibition of IBC tumor emboli cell growth includes the reduction or inhibition of IBC tumor cell proliferation, the reduction or inhibition of 3D IBC tumor emboli formation, and includes, in some embodiments, an increase in IBC tumor emboli cell death leading to a reduction in the number of cells in IBC tumor emboli. In some embodiments, the inhibition of IBC tumor emboli cell growth includes complete elimination of the IBC tumor emboli (e.g., complete cell death of all the cells in the 3D IBC tumor emboli).

In some embodiments, the at least one signal indicating IBC tumor emboli cell inhibition is selected from the group consisting of (i) a reduction in tumor emboli cell number, (ii) an increase in cell death, (iii) inhibition of tumor emboli spheroid formation, (iv) reduction of mitochondrial integrity, and (v) a combination thereof.

In some aspects, the signal indicating IBC tumor emboli cell growth inhibition is an increase in cell death and step (d) comprises incubating the 3D IBC spheroid array with at least one marker of cell death, wherein the an increase in the marker is an indicator of cell death.

Suitable markers of cell death are known in the art and include, but are not limited to, for example, YOYO-1, propidium iodide, Hoechst-33342, 4′, 6-diamidino-2-phenylindole (DAPI), annexin V, among others. In one embodiment, the marker of cell death is a fluorescent dye and the method comprises measuring the uptake of a fluorescent dye using high resolution imaging, wherein uptake of the fluorescent dye indicates cell death. Suitable fluorescent dyes include, for example, but not limited to, YOYO-1, among others.

In another aspect, the signal indicating IBC tumor emboli cell growth inhibition is measured using a cell viability marker. A cell viability marker may either indicated viable cells or may indicate dead cells. The marker allows for the quantitation of the live vs. dead cells in a sample. Suitable cell viability markers are known in the art and include, among others, the markers of cell death indicted above, MTS, XTT, and WST-1, annexin V, among others.

In some embodiments, the signal indicating IBC tumor emboli cell growth inhibition in reduction of mitochondrial integrity and wherein step (d) comprises incubating the 3D IBC spheroid array with a marker of mitochondrial integrity. Suitable markers of mitochondrial integrity are known in the art and include, but are not limited to Mitotracker™ Red, MTT, among others.

In a suitable embodiment, the at least one signal is at least two signals, preferably at least three signals. In one embodiment, the at least three signals are selected from the group consisting of (i) a reduction in tumor emboli cell number, (ii) an increase in cell death, (iii) inhibition of spheroid formation, (iv) reduction of mitochondrial integrity, and (v) a combination thereof

As discussed in the Examples below, the 3D tumor emboli assay described herein can be used to screen for the ability of anti-XIAP and anti-NFκB compounds to be used to inhibit IBC tumor emboli formation. Suitable anti-XIAP and anti-NFκB compounds are known in the art.

The assay described herein, in one embodiment, can be used for cancer phenotypic studies. Suitably, the combination of use of the three markers, YOYO-1, Hoechst and Mitotracker Red are optimized for use in the present invention.

In one embodiment, the method of identifying an agent able to inhibit tumor emboli cell growth includes incubating the 3D IBC spheroid array with Hoechst, YOYO-1, and Mitotracker Red in a single well of the array and measuring Hoechst, YOYO-1, and Mitotracker Red staining as an indication of cell morphology, cell viability, and mitochondrial function, respectively.

In yet another embodiment, step (d) comprises incubating the 3D IBC spheroid array with at least one fluorescent marker and using a microscope to image the array, wherein changes in fluorescence in the images compared to the non-contacted control incubated with the at least one fluorescent marker indicate IBC tumor emboli cell growth inhibition. In some embodiments, the fluorescent intensity of the sample is quantitated by comparing to a well not treated with the agent. Methods of quantitating fluorescence from images are known in the art. The images of the contacted wells are quantitated as compared to non-contacted control wells.

In some embodiments, once an agent is identified as able to inhibit IBC tumor emboli formation, the agent may be used to treat a subject having IBC. Suitable dosages will depend on a number of factors, including the age and health of the patient to be treated and would be able to be determined by the physician treating the subject.

The term “subject” and “patient” are used herein interchangeably and refer to a mammal, preferably a human, preferably a human having IBC.

In another embodiment, the present disclosure provides a kit comprising an in vitro 3-dimensional Inflammatory Breast Cancer Tumor Emboli high content throughput assay comprising: a) an array of 3-dimensional IBC tumor emboli within multiple culture wells; b) at least one marker able to produce a detectable signal, wherein the detectable signal is selected from the group consisting of (i) a reduction in tumor emboli cell number, (ii) an increase in cell death, (iii) inhibition of spheroid formation, (iv) reduction of mitochondrial integrity, and (v) a combination thereof; c) a microscope for imaging the detectable signal, and d) means for quantitatively analyzing the images.

Suitable means for quantitatively analyzing the images are known in the art, and include, but are not limited to, computer software able to process the images.

In some embodiments, the present invention provides methods of treating a subject having IBC, the method comprising identifying an agent able to inhibit IBC tumor emboli formation, and administering the agent in an effective amount to the subject to inhibit, reduce, or eliminate IBC tumor emboli within the subject.

The present invention has been described in terms of one or more preferred embodiments, and it should be appreciated that many equivalents, alternatives, variations, and modifications, aside from those expressly stated, are possible and within the scope of the invention.

The invention will be more fully understood upon consideration of the following non-limiting examples.

### EXAMPLES

### Example 1: Targeting and High Content, High-Throughput Assay for Characterization of 3D Inflammatory Breast Cancer Tumor Emboli Grown in Culture

The Example demonstrates for the first time a high content assay (HCA) using a single step staining protocol for simultaneously characterizing and quantitating multiple cell health characteristics of 3D IBC tumor emboli.

IBC pathobiology and disease progression is characterized by the presence of intralymphatic tumor emboli, which are postulated to contribute to dermal lymphatic invasion, collective migration and rapid metastatic progression (Vermeulen et al. 2010). There is a critical need for improved high-throughput methods to measure tumor emboli formation and toxicity in vitro to evaluate the potential effects of drugs and chemicals (industrial and environmental) during cancer progression and acquired therapeutic resistance. In addition, there is need for improved methods imaging and quantitation of the morphology of tumor cells comprising the spheroids. In this example, we show how IBC cell-derived tumor emboli can be used to evaluate morphological changes, spheroid formation, mitochondrial membrane potential and cell viability for drug screening using quantitative high content imaging assays that are also suitable for medium- to high-throughput.

Herein, we have discovered the optimization of a high content assay (HCA) using a single-step staining protocol to simultaneously characterize and quantitate multiple cell health characteristics of 3D IBC tumor emboli and the individual cells within these spheroids, as well as showcase the method's application in testing anticancer compounds in SUM149 and an isotype-derived drug resistant cell line, rSUM149 and in SUM190 IBC cells. While high content imaging of breast and colorectal cancer 3D spheroids has previously been shown (Harma et al. 2014; Reid et al. 2014; Trumpi et al. 2015; Wenzel et al. 2014), this is the first method, to the best of our knowledge, to combine a high-throughput multiparametric HCA with methods to generate 3D IBC spheroids. The extension of HCAs to 3D IBC tumor emboli has the potential to reduce cost and increase throughput of experiments by generating multiple quantitative parameters using automated imaging analysis. This method has the ability to reveal molecular and phenotypic determinants of this understudied cancer subtype and can be used to screen for inhibitors of spheroid formation using indicators of cell morphology and viability from a simple workflow assay protocol. Using known anticancer compounds in the cell lines tested, we demonstrated how multiple parameters can be used in combination to characterize adverse effects on spheroid formation, viability, proliferation, mitochondrial potential, and other parameters to assess cytotoxicity. This method can be used to gain insights into the methods sensitivity and specificity. While this example characterized treatment effects prior to spheroid formation, this model can also be extended to evaluate efficacy of various approved drugs and combinations during the tumor emboli growth at days 3 or 6 based on our current assay optimization data.

In summary, the IBC tumor emboli model combined with high content imaging-based assays presented in this study shows promise as a sensitive and reproducible tool for assessing changes in spheroid formation, viability and morphology. This model may be used for an in vitro system for screening of potential anticancer compound libraries, combinations of drug modalities, and effect of environmental chemicals.

### Materials and Methods

Cell Lines and Materials

SUM149 were obtained from Asterand, Inc. (Detroit, Mich.) and cultured in Ham's F-12 (Invitrogen, Carlsbad, Calif.)+5 μg/mL insulin+1 μg/mL hydrocortisone (Sigma-Aldrich, St. Louis, Mo.)+10 mM HEPES (Invitrogen)+5% FBS (not heat inactivated—Denville Scientific, South Plainfield N.J.) as described previously (Aird et al. 2008). rSUM149, a clonal population of acquired therapeutically resistant isotype-matched cell line derived from SUM149 (Aird et al. 2010) was cultured in SUM149 media supplemented with 7.5 μM of a research-grade lapatinib analog GW583340 (Williams, 2013). Asterand routinely characterizes cell lines by short tandem repeat polymorphism analysis. Cells were cultured in growth medium at 37° C. under an atmosphere of 5% CO2, were banked upon receipt and cultured for no more than 6 months prior to use in this study. All compounds (GANT61, Embelin, JSH-23) were purchased from Sigma Aldrich.

In Vitro Propagation of 3D IBC Tumor Spheroid and Emboli

2D cultured cells were dissociated as a single-cell suspension using trypsin and cell viability was determined by trypan blue exclusion as described previously (Aird et al. 2008), wherein % viability was calculated as live cells/total cells×100.

Spheroids in Ultra Low Attachment Plate

Single-cell suspensions of SUM149 and rSUM149 cells were plated in 6-well (40,000 cells/well), 12-well (20,000 cells/well) or 96-well (500-5,000 cells/well) ultra-low attachment plates (Corning, Glendale, Ariz.) in serum-free minimum essential media (MEM) (Invitrogen) supplemented with 20 ng/mL basic FGF, 20 ng/mL EGF and B27 (1× Invitrogen) for these experiments. 96-well experiments were conducted using black-walled, optical bottom, ultra-low attachment plates (Corning) for high content analysis. Treatments with compounds were carried out at the time of cell seeding in the ultra-low attachment plates, and spheroids were grown for up to 8 days (as indicated). Each compound was dissolved in dimethyl sulfoxide (DMSO) as a stock and treatment concentrations were based on previously published data regarding cytotoxicity of the compounds in SUM149 cells (Williams et al. 2013).

IBC Emboli Culture System Using the Belly Dancer Shaker Based on Lehman Et. Al. Int J Canc, 2012

Media: 149 media (Ham's F-12, with glutamine, 5% FBS, 1% P/S and hydrocortisone; only difference is the use of insulin/transferrin/selenium cocktail [Gibco], which we usually add separately to 190 media)

Flasks: Grenier CELLSTAR suspension flasks (we have one case already unopened)

Suspension liquid: 5 mL added to each flask containing 100 k single cells trypsinized from normal plates and 2.25% PEG8000 or 0.062% hyaluronic acid (HA) and growth medium

PEG8000 mammospheres grew slightly faster (Sigma 89510—based on Naigamwalla et. al. Cancer Res. 2000)

HA containing medium is more physiologically relevant as it is an endogenous component of lymphatic fluid.

Both compounds still exhibited similar properties to patient tumor emboli

Conditions: Flasks placed on Belly Dancer shaker in incubator, at 40 rpm and incubated in suspension for up to 96 h.

Microscopy

Phase contrast microscope images (4× or 10×) were taken daily up to 8 days to assess 3D spheroid and emboli formation using a Motic AE2000 microscope, M14 camera, and Infinity Capture (Lumenera, Ottawa, Canada) software.

Novel Tumor Emboli-High Content Multiparametric Assay:

To each well of the IBC tumor emboli growing in 6-well, 12-well, or 96-well plate (values for each plate type shown respectively), treatment medium was manually removed from each well with an automated 16-channel pipette, followed by addition of 960 μL, 480 μL, or 60 μL per well of pre-warmed dye cocktail in PBS (10 μg/mL Hoechst 33342, YOYO-1, 500 nM MitoTracker Red FM). Plates were then returned to the incubator for 45 minutes followed by removal of the 960 μL, 480 μL or 60 μL dye cocktail per well, and addition of 3200 μL, 1600 μL or 200 μL warmed PBS addition. Cells were then fixed after removing PBS and adding 960 μL, 480 μL or 60 μL of 10% formalin and incubated for 15 min at room temperature (RT) protected from light. 960 μL, 480 μL or 60 μL of formalin was then replaced with 3200 μL, 1600 μL or 200 μL of PBS prior to sealing the plates and imaging. Fluorescence quantification and localization was determined using a ThermoFisher Celllnsight NXT and 3-channel Cell Health Profiling protocol in HCS Screen software (ThermoFisher). Excitation wavelengths used were 386 nm, 485 nm, and 549 nm for Hoechst 33342, YOYO-1, and MitoTracker Red FM, respectively. Fixed exposure times were optimized in each channel for each experiment and set so that camera pixel intensity saturation was not reached. Images were then acquired using an Olympus UPlanFLN 10×/0.30 objective and 2×2 camera binning.

Statistical Analysis:

Quantitative data were expressed as mean±SEM. The statistical analyses were conducted using Graphpad Prism (Graphpad Software, Inc.) student's 2-tailed t-test and One way Anova Tukey Multiple comparison test and differences were considered significant at p<0.05.

### References for Example 1

Alpaugh M L, Tomlinson J S, Shao Z M, Barsky S H. 1999. A novel human xenograft model of inflammatory breast cancer. Cancer research 59(20): 5079-5084.
Lehman H L, Dashner E J, Lucey M, Vermeulen P, Dirix L, Van Laere S, et al. 2013. Modeling and characterization of inflammatory breast cancer emboli grown in vitro. International journal of cancer Journal international du cancer 132(10): 2283-2294.
Mu Z, Li H, Fernandez S V, Alpaugh K R, Zhang R, Cristofanilli M. 2013. EZH2 knockdown suppresses the growth and invasion of human inflammatory breast cancer cells. Journal of experimental & clinical cancer research: CR 32: 70.
Robertson F M, Bondy M, Yang W, Yamauchi H, Wiggins S, Kamrudin S, et al. 2010. Inflammatory breast cancer: the disease, the biology, the treatment. C A: a cancer journal for clinicians 60(6): 351-375.
Robertson F M, Chu K, Fernandez S V, Mu Z, Zhang X, Liu H, et al. 2012. Genomic Profiling of Pre-Clinical Models of Inflammatory Breast Cancer Identifies a Signature of Epithelial Plasticity and Suppression of TGFβ Signaling. J Clin Exp Pathol 2(5): 2161-0681.1000119.
Vermeulen P B, van Golen K L, Dirix L Y. 2010. Angiogenesis, lymphangiogenesis, growth pattern, and tumor emboli in inflammatory breast cancer: a review of the current knowledge. Cancer 116(11 Suppl): 2748-2754.
Wenzel C, Riefke B, Grundemann S, Krebs A, Christian S, Prinz F, et al. 2014. 3D high-content screening for the identification of compounds that target cells in dormant tumor emboli regions. Experimental cell research 323(1): 131-143.
Williams K P, Allensworth J L, Ingram S M, Smith G R, Aldrich A J, Sexton J Z, et al. 2013. Quantitative high-throughput efficacy profiling of approved oncology drugs in inflammatory breast cancer models of acquired drug resistance and re-sensitization. Cancer letters 337(1): 77-89.
Yamauchi H, Woodward W A, Valero V, Alvarez R H, Lucci A, Buchholz T A, et al. 2012. Inflammatory breast cancer: what we know and what we need to learn. The oncologist 17(7): 891-899.

### Example 2: Targeting Metastatic Dissemination in Inflammatory Breast Cancer

Effects of Oxidative Stress on Recurrent Tumor Cells Leading to TE Formation and Progression.

Using our novel, high content 3D TE in vitro models, we will (a) image and conduct quantitative assessment of the effect of oxidative stress stimuli on TE formation and individual cell health parameters in the TE; (b) characterize invasion, migration of individual tumor cells within the in vitro TE; (c) evaluate NFκB activation pathway expression in oxidative stress-induced survival signaling in recurrent tumor cells.

RTC/TE 3D Culture Model:

Therapy-resistant residual tumor cells evade programmed cell death/apoptosis and by clonal expansion give rise to RTC. In IBC, RTC form specialized tumor cell clusters, called tumor emboli (TE). These TE then migrate through the lymphatic system and spread to distant organs. A critical challenge is the characterization of individual cells that form TE to elucidate how they avoid apoptosis. The high-content assay (HCA) that utilizes combinations of nuclear and mitochondrial dyes as described in Example 1, which is the first quantitative assay developed for simultaneous analysis of multiple cell health indicators of whole 3D TE and the cells within (FIG. 2). Importantly, TE formation in IBC allows for invasion of the local dermal lymphatic vessels, promoting rapid systemic metastasis (Lehman, 2013). To recapitulate this in vitro, we have combined the HCA TE assay with a model that uses cells growing in a polyethylene glycol (PEG)- or hyaluronic acid (HA)-containing medium (this mimics the viscosity of lymphatic fluid) in a specialized shaker that simulates the oscillatory fluid shear forces present in in vitro lymphatics (Lehman, 2013). This HCA-TE-Lymphatic simulation model has distinct advantages: provides the closest approximation of the in vitro lymphatic milieu, where emboli invasion occurs simultaneously images and quantitative measures of TE and tumor cells comprising the TE, which can also be analyzed for single cell gene expression studies; and incorporates high-throughput liquid handling allowing for large-scale dose- & time-response screening before selecting the top candidates for in vivo evaluation

We will use western immunoblot analysis, immunofluorescence and/or NFκB-GFP reporter cell lines to assay for NFκB and target gene activation. HIF1 activation will serve as an NFκB activation marker when using the HIF-GFP reporter line or in assays testing protein expression (Gorlach, 2008). We will perform in vitro oxidative stress response measurements by directly monitoring ROS accumulation pre- and post-treatment by H₂DCFDA flow cytometry (Aird, 2012). MitoTracker Red staining in HCA-TE assay will also allow us to quantitate mitochondrial membrane potential, an important feature during oxidative stress.

Experiment 1A:

Live cell imaging and quantitative assessment of the effect of oxidative stress stimuli on TE formation and individual cell health parameters in TE.

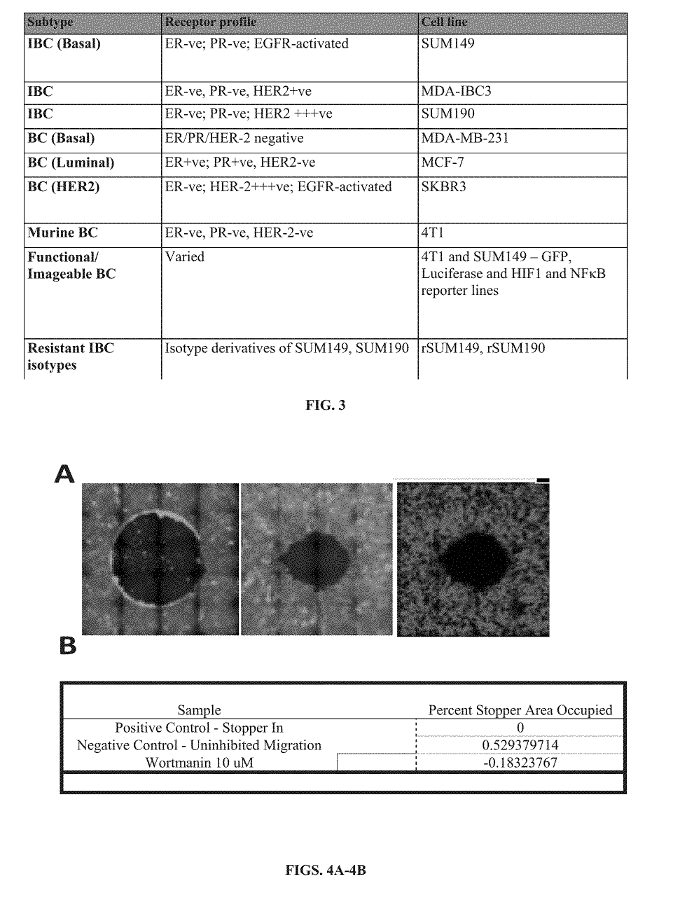

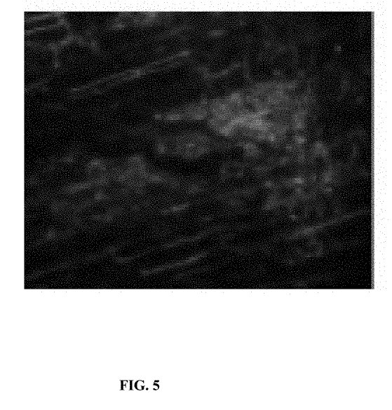

While SUM149, SUM190, MDAIBC3 cells form true TE-like clusters (FIG. 2C), most other BC cells can form mammospheres in culture using ultra-low attachment plates and specialized media, which we will test using the HCA system (FIG. 2) to study multiple cell lines in a high-throughput manner. IBC and nonlBC tumor cells (FIG. 3) will be either irradiated (2-15 Gy) or treated with doxorubicin, cyclophosphamide, docetaxel or lapatinib (0-10 μM) to generate dose- and time-response (0-10 day) data.

Experiment 1B.

IBC cell lines will also be used in the lymphatic simulation model (3D TE) to assess formation in this particular matrix (nonlBC cells do not form TE in this system). We will also determine the ability of preselected RTC cell populations (FIG. 3, Table 1) to form TE at basal and post RT/CT. For both Expt. 1A and 1B, two treatment models are proposed: 1) Pretreated 2D cells will be assessed for viability by trypan blue and equal numbers of viable cells from each treatment will be seeded in the 3D TE model to allow TE formation; 2) Pre-formed emboli will be treated to evaluate the effect of therapy in 3D culture. In addition to TE formation experiments, all treatments will be conducted in parallel 2D adherent experiments for comparison of endpoints.

The number of mammospheres (FIG. 2A) and TE (FIG. 2C) formed in the treated cell lines will be compared to the control groups. Quantitative data will be generated from the HCA analysis for nuclear count, mammosphere/TE size, shape, texture, area, individual cell viability, and proliferation (FIG. 2F) and isolated for oxidative stress markers, gene and protein expression analysis.

Experiment 2: Characterize Migration and Invasion of the Tumors Cells In Vitro.

Experiment 2A.

BC cells will be irradiated or treated with doxorubicin, cyclophosphamide, docetaxel or lapatinib (based on IC₁₀, IC₅₀, IC₉₀ doses identified in Expt. 1A). The treated cells will be used in a high content migration assay we have optimized (FIG. 4), which allows us to analyze tumor cell migration along with viability and proliferation. This assay is superior to standard scratch wound assays as it is able to distinguish agents affecting cell migration from those also affecting viability.

Experiment 2B.

For 3D invasion experiments, IBC tumor cells will first be grown in lymphatic simulating media and treated as listed in 2A. After treatment, TE will be harvested by low speed centrifugation (˜400 rpm), and resuspended in undiluted Matrigel. This mixture will be coated on the underside of transwell inserts in a modified amoeboid movement assay (Lehman, 2013). Serum-free media will be added to the companion plate and growth media added to the top of the insert. After 24 h incubation, inserts will be gently aspirated and dried, followed by crystal violet staining and manual counting of TE clusters comprising >50 cells at 10×.

Results from these experiments will identify the effect of radiation and chemotherapeutic agents on the ability of cells surviving therapy to form TE, and will allow us to compare IBC and nonIBC RTC TE formation. These experiments will generate quantitative measures of cell health indicators in individual cells in the TE, invasion and migration potential of the recurrent tumor cells/TE and effect of the RTC/TE on in vitro lymphangiogenesis. These experiments will allow for rapid, economical screening and provide clarity on treatment dose, time, and efficacy parameters that can be used for the in vivo studies. Expression analysis of NFκB pathway, HIF1 activity and oxidative stress response measurements obtained will reveal functional correlations with TE growth, invasion, and migration.

### Example 3: Determining the Effects of RT/CT-Mediated Oxidative Stress on Tumor Cell Invasion, Tumor-Vessel Interactions and Lymphangiogenesis on In Vivo BC Models

Not to be bound by any theory, but residual tumor cells often survive treatment through compensatory oxidative stress-mediated survival signaling and serve as reservoirs for tumor recurrence, invasion, and metastasis.

Breast tumor cells expressing NFκB or HIF1 reporter constructs will be implanted under murine window chambers in transgenic mice with fluorescent (different wavelength) lymph vasculature. This will allow simultaneous longitudinal imaging and quantification by in vivo high-resolution structural illumination or confocal-intravital microscopy of a) tumor initiation, b) regional metastasis c) dermal lymphatic invasion d) lymphangiogenesis, e) NFκB/HIF-1 expression, and f) oxidative stress response in RTC post-RT/CT.

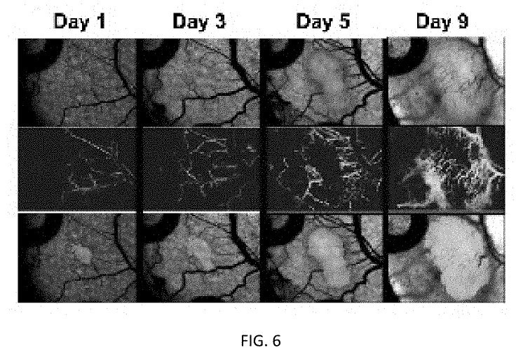

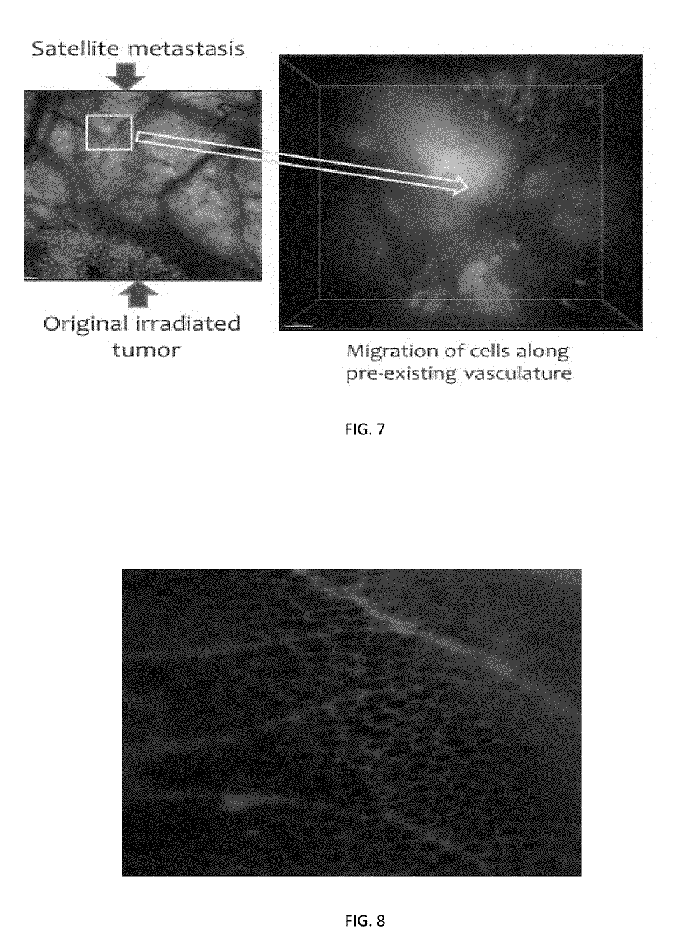

This approach (Palmer, 2011) involves surgical implantation of a titanium frame to support a glass window over the mouse's skin (FIG. 5) either in the dorsal or inguinal mammary fat pad sites. Fluorescently labeled tumor cells are then injected into the skin beneath windows implanted in BALB/c or nude mice (Moeller, 2004). High-resolution intravital microscopy is used to serially image the movement of cells (Betof, 2015; Cao, 2005; Li, 2000; Shan, 2004), thereby obtaining valuable multiparametric functional, molecular and quantitative information in vivo. RFP-tagged cells will enable us to visualize and quantify IBC cell infiltration into the lymphatics. This technique is beneficial in that we can image the same tumor and lymphatics for 7-10 days. Preliminary data show measurement of HIF1 levels, an NFκB target and biomarker for oxidative stress response using dual-tagged reporter cells (FIG. 6). Further, in a recent pilot study using a dorsal skin fold window chamber model, serial microscopic measurements were taken in live tumor-bearing mice before and after radiation exposure to show that tumor cells migrated along the external surface of tumor-associated vasculature adjacent to the irradiated tumor to the secondary site (FIG. 7). (Fontanelle, 2013; Hanna, 2013; Palmer, 2012; Palmer, 2011). These models enable high resolution, longitudinal monitoring of dynamic functional processes, which is not possible using other modalities. Of particular relevance, as shown in our data, we have applied such models to study oxidative stress as it is altered dynamically by therapy, while simultaneously assessing other parameters (e.g. angiogenesis, lymphangiogenesis, hypoxia, and tumor growth and migration).

Experiment 1: Determine Effect of Oxidative Stress on Regional Invasion

Experiment 1A.

We will use the inguinal mammary fat pad window chamber model, which allows for tumor growth in the natural microenvironment closer to the draining lymph nodes. 4T1 and SUM149 RFP/GFP reporter cell models will be implanted in the mammary window chamber model. When the tumors in the window reach a diameter of 2-4 mm post-implantation, we will acquire baseline-imaging data. Sentinel animals (5 mice/treatment/dose) will be treated with RT (5, 10 and 15 Gy) or doxorubicin (5, 10 mg/kg) systemically through tail vein injections to determine the optimal dose for induction of oxidative stress response. The single optimal doses of RT/CT will then be used for the remainder of the experiments, with 10 mice/group of the identified dose of RT and doxorubicin administered once. Longitudinal imaging of tumor cells and their movement will be performed using high-resolution intravital microscopy (Palmer, 2012; Palmer, 2011). Vascular length density (VLD) will be calculated to determine if there is a correlation between angiogenesis and metastasis, as we have previously reported that tumor-associated vasculature can provide a network for tumor cells to attach and move (Li, 2000). Hemoglobin saturation will be measured to provide a non-invasive assessment of oxygenation. This will aid in understanding if HIF-1 upregulation is influenced by hypoxia.

Experiment 1B.

To permit serial in vivo monitoring of tumor free-radicals, window chamber tumors will be suffused with media containing H₂DCFDA, which indicates the presence of free-radicals/oxidative stress. DCFDA, whose fluorescence is not free radical-responsive, will be used in a control group of tumors to rule out nonspecific radiation effects on dye accumulation.

Experiment 1C.

Resected tumors will be rendered transparent to visible light via optical-clearing and imaged using optical-CT/emission-CT (oCT/eCT) for HIF-1 or NFκB activation, vessel density, and regional migration/metastasis. Because the tumor cells constitutively express RFP or mCherry, which is not destroyed with optical-clearing, it is possible to follow these cells with fluorescence imaging and quantify the metastatic satellite tumor burden in these tissues with oCT/eCT (Oldham, 2008; Thomas, 2010).

Experiment 1D.

Approximately seven days after treatment and the completion of the longitudinal imaging series, tumors and surrounding normal tissue will be removed and assessed for signaling and oxidative stress response by measuring SOD activity, protein expression and total glutathione content. Immunohistochemistry, protein and RNA analysis will also be performed on a portion of the resected tumor. To further confirm the identity of recurrent tumor cells (RTC) post-RT/CT, SUM149 reporter cells that have migrated to the vasculature will be isolated and ALDH levels, a marker of stem-like cells, will be quantified using the ALDEFLUOR kit and remaining cells will subsequently be used in follow-up assays.

Experiment 2: Assessment of In Vivo Lymphangiogenesis.

For these experiments, we will utilize a ProxTom lymphatic vessel reporter B6 mouse (Truman, 2012), which has a TdTomato reporter present in the lymphatic system, backcrossed to nude mice. Through the use of the Duke breeding core facility that has expertise generating cross-strains of different mice, we will select for nude mice exhibiting the reporter at each generation (up until the 8th generation), which by then will have allowed for complete backcrossing. SUM149-GFP cells will be implanted during insertion of the mammary window chamber and treated once with the dose of RT or doxorubicin that gave the highest oxidative stress response used in Example 3—Expt. 1A (10 mice/group), which will allow us to track the invasion of tumor cells into the lymphatic vasculature.

For Expt. 2, similar quantitative measurements as elaborated in the window chamber model in Example 3—Expt. 1 will be carried out in vivo using high-resolution intravital microscopy (Palmer, 2012; Palmer, 2011). Lymph vessel length density (LVLD) will be calculated to assess correlation between lymphangiogenesis and metastasis.

Expected results (Table 3 summary of in vivo studies) will allow us to visualize and quantify in in vivo pre-clinical models the local migration induced by RT/CT-mediated oxidative stress. Through use of reporters, ROS-specific dyes, and endpoint analyses, we can correlate migration endpoints with oxidative stress response. Further, using unique lymphatic reporter systems, we can elucidate tumor cell interaction with lymphatic vessels to address the key feature of IBC cells: ability to undergo dermal, intralymphatic invasion and lymphangiogenesis. We expect our data will show enhanced migration, invasion and regional metastases after RT/CT through an increase in oxidative stress response, particularly activation of NFκB and downstream HIF1. We also expect to identify additional downstream proteins that are upregulated during RT/CT-mediated oxidative stress response, which will allow us to find new targets to prevent tumor recurrence.

TABLE 2
Summary of 4T1, and SUM149 Murine Window Chamber and
Orthotopic Tumor Models in Aim 3

| | | Combination | Endpoints | |
|---|---|---|---|---|
| Aim 3 (in vivo) | Treatment | (+/−) | (Primary, Secondary) | Mice |
| Exp. 2 - Short-term | Vehicle | RT* | Metastasis | N = 10/gp |
| murine window | DSF + Cu^{$} | Doxorubicin* | NFκB/HIF-1 | |
| chamber model for | MnP^{$$} | | VLD | |
| assessment of | Didox^{$$$} | | ROS levels | |
| treatment on invasion | | | Lymphangiogenesis | |
| and satellite | | | Endpoint (mRNA, | |
| metastasis [HIF1 and | | | protein, activity) | |
| NFκB reporter cells] | | | Locoregional failure | |
| | | | (tumors resected | |
| | | | from 5 mice/group | |
| | | | post RT/CT) | |
| Exp. 3 - Determination | Vehicle, RT* | Most efficacious | Tumor growth at site | N = 15/gp |
| of treatment strategies | Doxorubicin* | treatments from | of implantation | |
| on long-term | Docetaxel** | Exp 2 and single | Local and distant | |
| orthotopic mammary | Lapatinib*** | agents from | secondary metastasis | |
| tumor growth delay | DSF + Cu^{$} | Expt. 3 will | | |
| and metastatic model | MnP^{$$} | inform | | |
| [Luciferase cells] | Didox^{$$$} | combination | | |
| | | strategies | | |

*Optimum doses determined by sentinel animals
**6 mg/kg,
***100 mg/kg twice daily,
^{$}60 mg/kg DSF + 0.5 mg/kg Cu,
^{$$}0.2 mg/kg loading, 0.1 mg/kg 3 times/wk
^{$$$}425 mg/kg daily (IP)

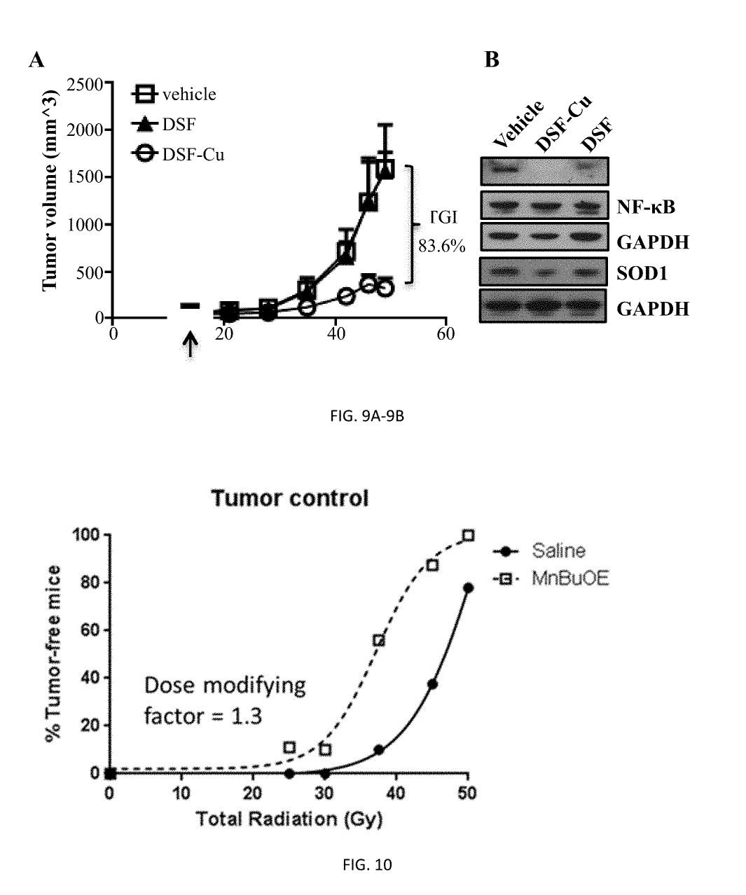

In an alternative experiment, we can use the traditional intradermal injection of 125 kDa fluorescently-labeled dextran conjugate as shown in FIG. 9 (Kilarski, 2013). If necessary, we can also compare with an alternate 3 and 10 kDa fluorescently-labeled dextran intravenous injection (Sarkisyan, 2012).

### Example 4: Identify Oxidative Stress Response Modulating Strategies to Inhibit Recurrent Tumor Cells, Prevent Metastatic Progression, and Enhance IBC Tumor Cell Kill

Strategies that enhance cell death in recurrent tumor cells during definitive therapy of primary IBC tumors or inhibit chest wall recurrence will reduce the risk of failure to control local disease and lead to much better patient survival/outcomes.

Approach:

We will test the efficacy of three redox modulatory strategies—DSF+Cu, MnSOD mimic and Didox in 1) SUM149, rSUM149, and 4T1-HIF-1 and NFκB reporter cells for their ability to suppress in vitro TE formation; 2) prevent dermal invasion, lymphangiogenesis in the in vivo window chamber models; 3) the oxidative stress response strategy showing the most potent efficacy in the in vitro TE assays and window chamber models will be extended to test in SUM149-, and 4T1-Luc tumors to suppress tumor growth and secondary local and distant metastasis as a single agent and in combination with RT and selected CT.

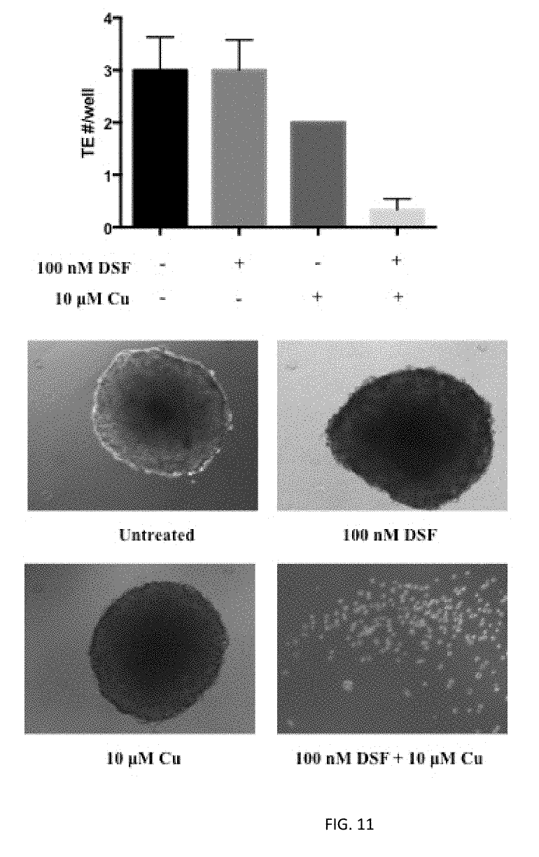

Work from our lab has shown comparison of orthotopic mammary fat pad tumors arising from a PTC line (SUM149 tested) to matched RTC derivative (rSUM149), revealing rSUM149 had enhanced tumor growth and secondary local and distant organ (lung) metastasis that mimics the morphology of TE (Lehman, 2013). Further, rSUM149 tumors similar to in vitro and gene expression analysis of post-treatment patient tumors had high oxidative stress response markers [XIAP, NFκB and NFκB targets (anti-apoptotic protein Bcl-2 and antioxidant SOD2)]. Recently, our lab has reported preclinical studies with DSF in IBC models (multiple PTC and RTC tested) and identified that DSF acts as a copper ionophore by bypassing the need for membrane transporters to induce copper-dependent oxidative stress selectively in tumor cells, suppressing NFκB activation and mediating anti-tumor efficacy (representative data in FIG. 10 (Allensworth, 2015)). Other data has shown that a MnSOD mimic abrogates RT-induced oxidative stress in a murine window chamber model, yet the addition of a MnSOD mimic sensitizes xenograft tumors to RT by a dose modifying factor of 1.3 (FIG. 11).

Experiment 1: Effect of Treatment Strategies on In Vitro Tumor Emboli Formation.

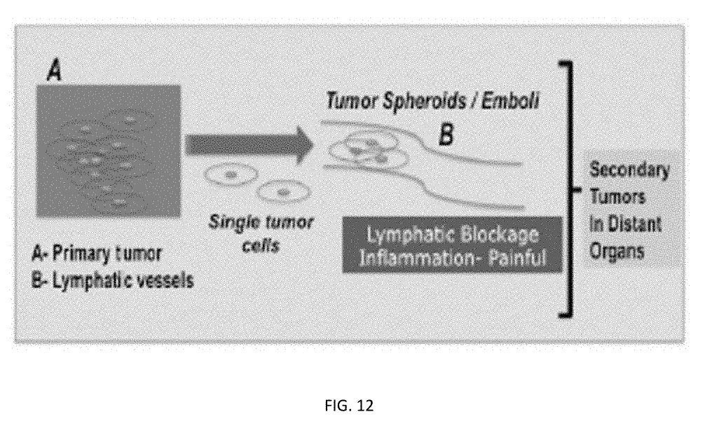

We will test the in vitro efficacy of redox modulating agents, DSF-Cu, MnSOD mimic, Didox (alone and in combination with RT/CT) to suppress in vitro TE formation and elucidate oxidative stress response and signaling. We will initially employ SUM149, rSUM149, and 4T1-HIF-1 and NFκB reporter cells (study design similar to protocols explained in Example 2—FIG. 12).

Experiment 2: Short-Term Murine Window Chamber Model for Assessment of Treatment on Invasion and Satellite Metastasis.

This experiment will determine in vivo efficacy of the redox modulating agents (alone and in combination with RT or doxorubicin) for their ability to suppress dermal invasion and lymphangiogenesis in mammary fat pad window chamber models.

Experiment 3: Determination of Treatment Strategies on Long-Term Tumor Growth Delay and Metastatic Model.

The in vivo efficacy of the most potent oxidative stress response-targeting agent alone and in combination with RT and select CT will be quantified in a murine orthotopic mammary tumor growth model. In this experiment, the strategy that shows the most potent efficacy in the in vitro TE assays and the window chamber models will be extended to test in SUM149-, and 4T1-Luc in vivo tumors to suppress tumor growth and secondary local and distant metastasis as a single agent and in combination with RT and selected CT. SUM149-luc and 4T1-luc will be implanted orthotopically (5×10⁶ cells in 50 μL) in the fourth mammary fat pad of nude or BALB/c mice, respectively. When the tumors reach 100 mm³, treatment groups (15 mice/group) will include: 15 Gy RT (once), 10 mg/kg doxorubicin (once/week, iv, tail vein), 6 mg/kg docetaxel (once/wk, ip), 100 mg/kg lapatinib (twice daily, po) (Kurokawa, 2013), 50 mg/kg of DSF+0.5 mg/kg Cu (daily, ip), MnP (loading dose of 0.2 mg/kg, followed by maintenance dose of 0.1 mg/kg 3 days/week), 425 mg/kg didox (daily, ip) and appropriate vehicle treated mice groups. Results from Example 3 and single agent studies in this aim will also inform us about optimal dose and combinations for further testing in the tumor models. Longitudinal luciferase imaging of both primary tumors and distant metastases will be conducted every 3 days using a Xenogen IVIS Lumina XR system. Seven days post-treatment, a set of mice will have their primary tumors surgically removed and be followed for secondary tumors (local dermal and distant organ) to mimic a clinical regimen of RT/CT and then surgery. Tumor volume [(length×width²)/2] in the remaining mice will be monitored until tumors reach 1500 mm³ or mice show signs of morbidity. The fold-change in tumor volume will be normalized to baseline size and plotted over the indicated points to generate tumor growth graphs using GraphPad Prism. Enhancement ratios will be determined by dividing the average tumor volumes of tumors receiving RT/CT alone by those receiving RT/CT in combination. Distant metastases will be monitored using the IVIS Lumina XR system. In addition, optical-clearing and imaging via oCT/eCT of lung tissues will be performed to quantify the extent of lung metastasis (Oldham, 2008; Thomas, 2010). Vascular length density will also be quantified. Resected tumors will be used to assay for oxidative stress along with other parameters derived in previous RNA/protein analyses. Excised tumors will be processed for H&E staining (to assess tumor differentiation); immunohistochemical analysis of NFκB, XIAP, SOD1 and HIF1 and proliferation index (Ki67). Epithelial, mesenchymal and stem-like markers (ALDH+ and CD24−,CD44+ tumor cells) will be evaluated in the primary tumor and secondary metastasis/TE samples as previously described for IBC samples (Robertson, 2012).

Experiment 4: Elucidate Biomarkers for Monitoring Oxidative Stress Response In Vivo.

Measurement of ROS in vivo carries a significant analytical challenge, as most ROS are highly reactive and short lived, making it difficult to detect directly. Furthermore, it is important to assess oxidative damage at both systemic and tissue-specific levels. The systemic levels of the biomarkers reflect individual oxidative status that may be involved in creating important environmental factors for tumor development and response to treatment (Il'yasova, 2011). The oxidative status of tumor tissue reflects interaction between cellular redox balance and response to treatment. Frequently used biomarkers of oxidative stress, such as protein carbonyl groups and malondialdehyde, are not specific to any particular oxidative processes and have been shown to be unresponsive markers in animal and clinical models of oxidative stress (Halliwell, 2004; Il'yasova, 2009; Il'yasova, 2010; Kadiiska, 2005). Major principles in selecting biomarkers for this study are: A. chemically stable oxidative modification, B. measurable in non-cancer patients and normal tissues, C. reflect ROS-specific chemical modifications. We will use the following markers in tissue, or in peripheral blood samples collected from all the in vivo studies as applicable. 1) SOD1/2 activity in tumor tissue: We will use a superoxide generating system (xanthine and xanthine oxidase) to measure SOD1 and SOD2 enzyme activities. We will quantify the ability of increasing amounts of tumor tissue lysate to inhibit the reduction of NBT to blue formazan (Spitz, 1989). 2) GSH in blood: We will measure depletion of total glutathione and a decreased GSH/GSSG ratio in peripheral blood samples from the tumor studies by HPLC (Rossi, 2006). 3) Immunohistochemistry: We will collaborate with Dr. Hwang and the Duke Cancer Center Histology Core to conduct expression of XIAP, Smac, SOD2, and thioredoxin-1 in tumor tissue.

Expected Results:

We anticipate identification of potent combinations that will be superior to single agents in inhibition of tumor growth and/or metastasis. Table 2 also summarizes the in vivo animal studies related to this Aim 3. Further, DSF is an inhibitor of ALDH, also a marker of tumor stem-like cells and because TE are rich in ALDH1+ve cells, we anticipate DSF-Cu to be effective in inhibition of TE formation as observed in pilot studies (FIG. 12). We expect to identify important oxidative stress response-related biomarkers that can be measured in biopsies and peripheral blood samples and correlate with therapeutic response.

TABLE 3
Summary of 4T1 and SUM149 Murine Window Chamber Tumor
Model

| | | Endpoints | |
|---|---|---|---|
| | | (Primary, | |
| Aim | Treatment | Secondary) | Mice |
| Exp. 1a - | Vehicle | Tumor ROS | N = 5/gp |
| Determination of | RT (5, 10, 15 Gy) | (H₂DCFDA) | |
| RT/CT dosing for | Doxorubicin | | |
| oxidative stress | (5, 10 mg/kg) | | |
| [Sentinel Animals] | | | |
| Exp. 1b, c, d - | Vehicle | Regional invasion, | N = 10/gp |
| Effect of oxidative | RT (Optimum | migration & | |
| stress on regional | dose from sentinel | metastasis | |
| invasion [HIF1 | animals) | NFκB/HIF-1 levels | |
| and NFκB reporter | Doxorubicin | VLD | |
| cell lines] | (Optimum dose from | ROS and stress | |
| | sentinel animals) | response | |
| Exp. 2 - Assess in | Vehicle | See Exp. 1 | N = 10/gp |
| vivo | RT (selected dose | Endpoint (mRNA, | |
| lymphangiogenesis | from above) | protein, activity) | |
| [GFP cell lines] | Doxorubicin | | |
| | (selected dose | | |
| | from above) | | |

Potential Pitfalls/Alternate Approaches:

Although the NFκB pathway is key in the oxidative stress response, we recognize this may not be the only mechanism that can solely inform on all aspects of tumor recurrence and dermal lymphatic invasion in IBC. Based on our molecular data regarding how the anti-apoptotic protein, XIAP, interacts with NFκB in IBC cells (Evans, 2014a), future studies will evaluate strategies (Allensworth, 2012; Allensworth, 2015; Evans, 2014c) we have characterized to target XIAP, including: 1) embelin, a plant-derived small molecule inhibitor of the XIAP-caspase-9 interaction which has been shown to inhibit NFκB activation in multiple models of cancer (Ahn, 2007) and to enhance the efficacy RT in prostate cancer (Dai, 2011), and 2) smac mimetics, potent XIAP antagonists that induce apoptosis in BC cells as well as act as radiosensitizers when used in combination with RT (Yang, 2012). Additionally, if we do not see antitumor efficacy of MnP, we can employ the combination strategy of MnP+ascorbate, which we have recently shown in in vitro models can inhibit growth of both sensitive and resistant IBC cells through increased oxidative stress (Evans, 2014c). In the case of redox response markers, if thioredoxin-1 turns out to not be predictive, other thiol-based members of the cellular hydroperoxide metabolism system (namely peroxiredoxin-1 and glutatredoxin-1) will be investigated through IHC (Aesif, 2011; Hansen, 2007). We will extend analysis of these biomarkers by measuring F2-isoprostanes (Il'yasova, 2010), which can also be extended to test frozen urinary samples retrospectively collected from IBC patients. Currently, we are conducting IHC analysis of oxidative stress response biomarkers in archival tissue microarray (TMA) samples IBC, nonlBC and normal patient tissue obtained in collaboration with World IBC consortium and the Duke Inflammatory Breast Cancer Consortium.

Prior publications from our lab will also inform on analysis including EC50 calculations and evaluation of combination drug studies (Allensworth, 2012; Devi, 2005; Morse, 2010; Rangwala, 2012; Sekhon, 2008; Thomas, 2011). Image data obtained from intra-vital microscopy will be converted into quantitative measures to represent various aspects of tumor morphology and physiology. For instance, local metastasis will be quantified as a moment measure (M), a product of the size of regional metastasis and their distance from the main tumor. Angiogenesis will be quantified by serial measurements of vascular length density (VLD) and physiological measurements such as HIF-1 levels will be quantified by fluorescence intensity (F) of the HIF-1-GFP reporter. Serial measurements of these quantitative biomarkers will be analyzed across treatment groups using a mixed linear model (Fitzmaurice, 2012), which randomizes effects to account for variability within and across animals, as well as correlations between multiple biomarkers measured on the same animal. A similar modeling strategy will be used for in vitro experiments. Sample size justification: We focus on the hypothesis test: H0: λf=λp vs. H1: λf<λp, where λf and λp are the average number of regional metastases per animal in the full and respectively partially irradiated groups. Assuming a Poisson distribution for the observed number of regional metastases per animal, we propose to compute a two sample Poisson test (Zar, 2010). Assuming an average of 1 metastases in the partial group and 0.5 in the full group (conservative projection from preliminary data), at a 95% significance level, we obtain >80% power with 10 animals/group.

### References Cited in Examples 2-4

Abramowitz M C, Li T, Morrow M, Sigurdson E R, Anderson P, Nicolaou N, Freedman G. Dermal lymphatic invasion and inflammatory breast cancer are independent predictors of outcome after postmastectomy radiation. American journal of clinical oncology. 2009; 32(1):30-3. doi: 10.1097/COC.0b013e31817b6073. PubMed PMID: 19194121.
Abrous-Anane S, Savignoni A, Daveau C, Pierga J Y, Gautier C, Reyal F, Dendale R, Campana F, Kirova Y M, Fourquet A, Bollet M A. Management of inflammatory breast cancer after neoadjuvant chemotherapy. International journal of radiation oncology, biology, physics. 2011; 79(4):1055-63. doi: 10.1016/j.ijrobp.2009.12.009. PubMed PMID: 20478662.
Aesif S W, Kuipers I, van der Velden J, Tully J E, Guala A S, Anathy V, Sheely J I, Reynaert N L, Wouters E F, van der Vliet A, Janssen-Heininger Y M. Activation of the glutaredoxin-1 gene by nuclear factor kappaB enhances signaling. Free radical biology & medicine. 2011; 51(6):1249-57. Epub 2011/07/19. doi: 10.1016/j.freeradbiomed.2011.06.025. PubMed PMID: 21762778; PubMed Central PMCID: PMC3181077.
Ahn K S, Sethi G, Aggarwal B B. Embelin, an inhibitor of X chromosome-linked inhibitor-of-apoptosis protein, blocks nuclear factor-kappaB (NF-kappaB) signaling pathway leading to suppression of NF-kappaB-regulated antiapoptotic and metastatic gene products. Molecular pharmacology. 2007; 71(1):209-19. doi: 10.1124/mol.106.028787. PubMed PMID: 17028156.
Aird K M, Allensworth J L, Batinic-Haberle 1, Lyerly H K, Dewhirst M W, Devi G R. ErbB1/2 tyrosine kinase inhibitor mediates oxidative stress-induced apoptosis in inflammatory breast cancer cells. Breast cancer research and treatment. 2012; 132(1):109-19. Epub 2011/05/12. doi: 10.1007/s10549-011-1568-1. PubMed PMID: 21559822.
Aird K M, Ghanayem R B, Peplinski S, Lyerly H K, Devi G R. X-linked inhibitor of apoptosis protein inhibits apoptosis in inflammatory breast cancer cells with acquired resistance to an ErbB1/2 tyrosine kinase inhibitor. Molecular cancer therapeutics. 2010; 9(5):1432-42. doi: 10.1158/1535-7163.MCT-10-0160. PubMed PMID: 20406946; PubMed Central PMCID: PMC2957814.
Al-Abd A M, Al-Abbasi F A, Asaad G F, Abdel-Naim A B. Didox potentiates the cytotoxic profile of doxorubicin and protects from its cardiotoxicity. European journal of pharmacology. 2013; 718(1-3):361-9. doi: 10.1016/j.ejphar.2013.08.009. PubMed PMID: 24021537.
Allensworth J L, Aird K M, Aldrich A J, Batinic-Haberle 1, Devi G R. XIAP Inhibition and Generation of Reactive Oxygen Species Enhances TRAIL Sensitivity in Inflammatory Breast Cancer Cells. Molecular cancer therapeutics. 2012. Epub 2012/04/18. doi: 10.1158/1535-7163.MCT-11-0787. PubMed PMID: 22508521.
Allensworth J L, Evans M K, Bertucci F, Aldrich A J, Festa R A, Finetti P, Ueno N T, Safi R, McDonnell D P, Thiele D J, Van Laere S, Devi G R. Disulfiram (DSF) acts as a copper ionophore to induce copper-dependent oxidative stress and mediate anti-tumor efficacy in inflammatory breast cancer. Mol Oncol. 2015. doi: 10.1016/j.molonc.2015.02.007. PubMed PMID: 25769405.
Alvarez J V, Pan T C, Ruth J, Feng Y, Zhou A, Pant D, Grimley J S, Wandless T J, Demichele A, Investigators IST, Chodosh L A. Par-4 downregulation promotes breast cancer recurrence by preventing multinucleation following targeted therapy. Cancer cell. 2013; 24(1):30-44. doi: 10.1016/j.ccr.2013.05.007. PubMed PMID: 23770012; PubMed Central PMCID: PMC3808871.
Arora A J, Samal L, Tarpley M, Williams K P, Dewhirst M W, Devi G R, Sauer S J, editors. Development of a Novel High Content Multiparametric Assay for the Quantitative Analysis of Cell Health in Tumor Emboli in Culture 2nd Annual Duke Cancer Institute Scientific Retreat; 2014; Durham, N.C.
Aseervatham G S, Sivasudha T, Jeyadevi R, Arul Ananth D. Environmental factors and unhealthy lifestyle influence oxidative stress in humans—an overview. Environmental science and pollution research international. 2013; 20(7):4356-69. doi: 10.1007/s11356-013-1748-0. PubMed PMID: 23636598.
Bates T, Williams N J, Bendall S, Bassett E E, Coltart R S. Primary chemo-radiotherapy in the treatment of locally advanced and inflammatory breast cancer. Breast. 2012; 21(3):330-5. doi: 10.1016/j.breast.2012.02.002. PubMed PMID: 22410111.
Bertucci F, Ueno N T, Finetti P, Vermeulen P, Lucci A, Robertson F M, Marsan M, Iwamoto T, Krishnamurthy S, Masuda H, Van Dam P, Woodward W A, Cristofanilli M, Reuben J M, Dirix L, Viens P, Symmans W F, Birnbaum D, Van Laere S J. Gene expression profiles of inflammatory breast cancer: correlation with response to neoadjuvant chemotherapy and metastasis-free survival. Annals of oncology: official journal of the European Society for Medical Oncology/ESMO. 2014; 25(2):358-65. doi: 10.1093/annonc/mdt496. PubMed PMID: 24299959; PubMed Central PMCID: PMC3905779.
Betof A S, Lascola C D, Weitzel D, Landon C, Scarbrough P M, Devi G R, Palmer G, Jones L W, Dewhirst M W. Modulation of murine breast tumor vascularity, hypoxia and chemotherapeutic response by exercise. Journal of the National Cancer Institute. 2015; 107(5). doi: 10.1093/jnci/djv040. PubMed PMID: 25780062.
Bourgier C, Pessoa E L, Dunant A, Heymann S, Spielmann M, Uzan C, Mathieu M C, Arriagada R, Marsiglia H. Exclusive alternating chemotherapy and radiotherapy in nonmetastatic inflammatory breast cancer: 20 years of follow-up. International journal of radiation oncology, biology, physics. 2012; 82(2):690-5. doi: 10.1016/j.ijrobp.2010.11.040. PubMed PMID: 21277101.
Brown N S, Bicknell R. Hypoxia and oxidative stress in breast cancer. Oxidative stress: its effects on the growth, metastatic potential and response to therapy of breast cancer. Breast cancer research: BCR. 2001; 3(5):323-7. PubMed PMID: 11597322; PubMed Central PMCID: PMC138696.
Cao Y, Eble J M, Moon E, Yuan H, Weitzel D H, Landon C D, Nien C Y, Hanna G, Rich J N, Provenzale J M, Dewhirst M W. Tumor cells upregulate normoxic HIF-1alpha in response to doxorubicin. Cancer research. 2013; 73(20):6230-42. doi: 10.1158/0008-5472.CAN-12-1345. PubMed PMID: 23959856; PubMed Central PMCID: PMC3800255.
Cao Y, Li C Y, Moeller B J, Yu D, Zhao Y, Dreher M R, Shan S, Dewhirst M W. Observation of incipient tumor angiogenesis that is independent of hypoxia and hypoxia inducible factor-1 activation. Cancer research. 2005; 65(13):5498-505. doi: 10.1158/0008-5472.CAN-04-4553. PubMed PMID: 15994919.
Cheng Z X, Sun B, Wang S J, Gao Y, Zhang Y M, Zhou H X, Jia G, Wang Y W, Kong R, Pan S H, Xue D B, Jiang H C, Bai X W. Nuclear factor-kappaB-dependent epithelial to mesenchymal transition induced by HIF-1alpha activation in pancreatic cancer cells under hypoxic conditions. PloS one. 2011; 6(8):e23752. doi: 10.1371/journal.pone.0023752. PubMed PMID: 21887310; PubMed Central PMCID: PMC3161785.
Chu K, Boley K M, Moraes R, Barsky S H, Robertson F M. The paradox of E-cadherin: role in response to hypoxia in the tumor microenvironment and regulation of energy metabolism. Oncotarget. 2013; 4(3):446-62. PubMed PMID: 23530113; PubMed Central PMCID: PMC3717307.
Clemons M, Danson S, Hamilton T, Goss P. Locoregionally recurrent breast cancer: incidence, risk factors and survival. CANCER TREATMENT REVIEWS. 2001; 27:67-82.
Cook G J, Caudell D L, Elford H L, Pardee T S. The Efficacy of the Ribonucleotide Reductase Inhibitor Didox in Preclinical Models of AML. PloS one. 2014; 9(11):e112619. doi: 10.1371/journal.pone.0112619. PubMed PMID: 25402485; PubMed Central PMCID: PMC4234372.
Crawford D R, Edbauer-Nechamen C A, Lowry C V, Salmon S L, Kim Y K, Davies J M, Davies K J. Assessing gene expression during oxidative stress. Methods in enzymology. 1994; 234:175-217. PubMed PMID: 7528871.
Dai Y, Desano J, Qu Y, Tang W, Meng Y, Lawrence T S, Xu L. Natural IAP inhibitor Embelin enhances therapeutic efficacy of ionizing radiation in prostate cancer. American journal of cancer research. 2011; 1(2):128-43. PubMed PMID: 21804946; PubMed Central PMCID: PMC3144474.
Dawood S, Cristofanilli M. Inflammatory breast cancer: what progress have we made? Oncology (Williston Park). 2011a; 25(3):264-70, 73. Epub 2011/05/10. PubMed PMID: 21548470.
Dawood S, Ueno N T, Valero V, Woodward W A, Buchholz T A, Hortobagyi G N, Gonzalez-Angulo A M, Cristofanilli M. Differences in survival among women with stage III inflammatory and noninflammatory locally advanced breast cancer appear early: a large population-based study. Cancer. 2011b; 117(9):1819-26. doi: 10.1002/cncr.25682. PubMed PMID: 21509759.
Devi G R, editor. Determination of an oxidative stress gene signature in inflammatory breast cancer patient tumors and development of a novel redox modulatory strategy in overcoming chemotherapy resistance and mediating anti-tumor efficacy. AACR-EORTC Abstract; 2014a; Barcelona, Spain.
Devi G R, Allensworth J L, Evans M K, Sauer S J. The Role of Oxidative Stress in Breast Cancer. In: Preedy V R, editor. Cancer: Oxidative Stress and Dietary Antioxidants. London: Elsevier; 2014c.
Devi G R, Beer T M, Corless C L, Arora V, Weller D L, Iversen P L. In vivo bioavailability and pharmacokinetics of a c-MYC antisense phosphorodiamidate morpholino oligomer, AVI-4126, in solid tumors. Clinical cancer research: an official journal of the American Association for Cancer Research. 2005; 11(10):3930-8. PubMed PMID: 15897595.
Dewhirst M W, Cao Y, Li C Y, Moeller B. Exploring the role of HIF-1 in early angiogenesis and response to radiotherapy. Radiotherapy and oncology: journal of the European Society for Therapeutic Radiology and Oncology. 2007; 83(3):249-55. doi: 10.1016/j.radonc.2007.05.016. PubMed PMID: 17560674; PubMed Central PMCID: PMC2694841.
Evans M K, Sauer S J, Aldrich A J, Geradts J, Vermeulen P, Dirix L, Van Laere S, Devi G R, editors. A novel link between anti-apoptotic signaling, NFκB, and SMAD7 in IBC pathobiology. San Antonio Breast Cancer Symposium; 2014a; San Antonio, Tex.
Evans M K, Tovmasyan A, Batinic-Haberle I, Devi G R. Mn porphyrin in combination with ascorbate acts as a pro-oxidant and mediates caspase-independent cancer cell death. Free radical biology & medicine. 2014c; 68:302-14. doi: 10.1016/j.freeradbiomed.2013.11.031. PubMed PMID: 24334253.
Figul M, Soling A, Dong H J, Chou T C, Rainov N G. Combined effects of temozolomide and the ribonucleotide reductase inhibitors didox and trimidox in malignant brain tumor cells. Cancer chemotherapy and pharmacology. 2003; 52(1):41-6. doi: 10.1007/s00280-003-0611-2. PubMed PMID: 12690517.
Fitzmaurice G M, Laird N M, Ware J H. Applied longitudinal analysis: John Wiley & Sons; 2012.
Fontanelle A N, Schroeder T, Hochman D W, Chen R E, Hanna G, Haglund M M, Secomb T W, Palmer G M, Dewhirst M W. Quantitative mapping of hemodynamics in the lung, brain, and dorsal window chamber-grown tumors using a novel, automated algorithm. Microcirculation. 2013. doi: 10.1111/micc.12072. PubMed PMID: 23781901.
Forman H J, Maiorino M, Ursini F. Signaling functions of reactive oxygen species. Biochemistry. 2010; 49(5):835-42. doi: 10.1021/bi9020378.
Giampieri S, Manning C, Hooper S, Jones L, Hill C S, Sahai E. Localized and reversible TGFbeta signalling switches breast cancer cells from cohesive to single cell motility. Nature cell biology. 2009; 11(11):1287-96. doi: 10.1038/ncb1973. PubMed PMID: 19838175; PubMed Central PMCID: PMC2773241.
Gorlach A, Bonello S. The cross-talk between NF-kappaB and HIF-1: further evidence for a significant liaison. The Biochemical journal. 2008; 412(3):e17-9. doi: 10.1042/BJ20080920. PubMed PMID: 18498249.
Gorrini C, Harris I S, Mak T W. Modulation of oxidative stress as an anticancer strategy. Nature reviews Drug discovery. 2013; 12(12):931-47. doi: 10.1038/nrd4002. PubMed PMID: 24287781.
Gridley D S, Makinde A Y, Luo X, Rizvi A, Crapo J D, Dewhirst M W, Moeller B J, Pearlstein R D, Slater J M. Radiation and a metalloporphyrin radioprotectant in a mouse prostate tumor model. Anticancer research. 2007; 27(5A):3101-9. PubMed PMID: 17970050.
Halliwell B, Whiteman M. Measuring reactive species and oxidative damage in vivo and in cell culture: how should you do it and what do the results mean? British journal of pharmacology. 2004; 142(2):231-55. Epub 2004/05/25. doi: 10.1038/sj.bjp.0705776. PubMed PMID: 15155533; PubMed Central PMCID: PMC1574951.
Hanahan D, Weinberg R A. Hallmarks of cancer: the next generation. Cell. 2011; 144(5):646-74. doi: 10.1016/j.cell.2011.02.013. PubMed PMID: 21376230.
Hance K W, Anderson W F, Devesa S S, Young H A, Levine P H. Trends in inflammatory breast carcinoma incidence and survival: the surveillance, epidemiology, and end results program at the National Cancer Institute. Journal of the National Cancer Institute. 2005; 97(13):966-75. doi: 10.1093/jnci/dji172. PubMed PMID: 15998949; PubMed Central PMCID: PMC2844937.
Hanna G, Fontanelle A, Palmer G, Shan S, Radiloff D R, Zhao Y, Irwin D, Hamilton K, Boico A, Piantadosi C A, Blueschke G, Dewhirst M, McMahon T, Schroeder T. Automated measurement of blood flow velocity and direction and hemoglobin oxygen saturation in the rat lung using intravital microscopy. American journal of physiology Lung cellular and molecular physiology. 2013; 304(2):L86-91. doi: 10.1152/ajplung.00178.2012. PubMed PMID: 23161885.
Hansen J M, Moriarty-Craige S, Jones D P. Nuclear and cytoplasmic peroxiredoxin-1 differentially regulate NF-kappaB activities. Free radical biology & medicine. 2007; 43(2):282-8. Epub 2007/07/03. doi: 10.1016/j.freeradbiomed.2007.04.029. PubMed PMID: 17603937; PubMed Central PMCID: PMC2096473.
Hoshida T, Isaka N, Hagendoorn J, di Tomaso E, Chen Y L, Pytowski B, Fukumura D, Padera T P, Jain R K. Imaging steps of lymphatic metastasis reveals that vascular endothelial growth factor-C increases metastasis by increasing delivery of cancer cells to lymph nodes: therapeutic implications. Cancer research. 2006; 66(16):8065-75. doi: 10.1158/0008-5472.CAN-06-1392. PubMed PMID: 16912183.
Il'yasova D, Mixon G, Wang F, Marcom P K, Marks J, Spasojevich I, Craft N, Arredondo F, DiGiulio R. Markers of oxidative status in a clinical model of oxidative assault: a pilot study in human blood following doxorubicin administration. Biomarkers: biochemical indicators of exposure, response, and susceptibility to chemicals. 2009; 14(5):321-5. Epub 2009/05/30. doi: 10.1080/13547500902946757. PubMed PMID: 19476408; PubMed Central PMCID: PMC2716435.
Il'yasova D, Siamakpour-Reihani S, Akushevich I, Akushevich L, Spector N, Schildkraut J. What can we learn from the age- and race/ethnicity-specific rates of inflammatory breast carcinoma? Breast cancer research and treatment. 2011; 130(2):691-7. Epub 2011/08/19. doi: 10.1007/s10549-011-1719-4. PubMed PMID: 21850396.
Il'yasova D, Spasojevic I, Wang F, Tolun A A, Base K, Young S P, Marcom P K, Marks J, Mixon G, DiGiulio R, Millington D S. Urinary biomarkers of oxidative status in a clinical model of oxidative assault. Cancer epidemiology, biomarkers & prevention: a publication of the American Association for Cancer Research, cosponsored by the American Society of Preventive Oncology. 2010; 19(6):1506-10. Epub 2010/05/27. doi: 10.1158/1055-9965.EPI-10-0211. PubMed PMID: 20501773; PubMed Central PMCID: PMC2883001.
Inayat M S, Chendil D, Mohiuddin M, Elford H L, Gallicchio V S, Ahmed M M. Didox (a novel ribonucleotide reductase inhibitor) overcomes Bcl-2 mediated radiation resistance in prostate cancer cell line PC-3. Cancer biology & therapy. 2002; 1(5):539-45. PubMed PMID: 12496485.
Johansson B. A review of the pharmacokinetics and pharmacodynamics of disulfiram and its metabolites. Acta psychiatrica Scandinavica Supplementum. 1992; 369:15-26. PubMed PMID: 1471547.
Kadiiska M B, Gladen B C, Baird D D, Germolec D, Graham L B, Parker C E, Nyska A, Wachsman J T, Ames B N, Basu S, Brot N, Fitzgerald G A, Floyd R A, George M, Heinecke J W, Hatch G E, Hensley K, Lawson J A, Marnett L J, Morrow J D, Murray D M, Plastaras J, Roberts L J, 2nd, Rokach J, Shigenaga M K, Sohal R S, Sun J, Tice R R, Van Thiel D H, Wellner D, Walter P B, Tomer K B, Mason R P, Barrett J C. Biomarkers of oxidative stress study II: are oxidation products of lipids, proteins, and DNA markers of CC14 poisoning? Free radical biology & medicine. 2005; 38(6):698-710. Epub 2005/02/22. doi: 10.1016/j.freeradbiomed.2004.09.017. PubMed PMID: 15721980.
Kakehashi A, Wei M, Fukushima S, Wanibuchi H. Oxidative stress in the carcinogenicity of chemical carcinogens. Cancers. 2013; 5(4):1332-54. doi: 10.3390/cancers5041332. PubMed PMID: 24202448; PubMed Central PMCID: PMC3875942.
Kilarski W W, Guc E, Teo J C, Oliver S R, Lund A W, Swartz M A. Intravital immunofluorescence for visualizing the microcirculatory and immune microenvironments in the mouse ear dermis. PloS one. 2013; 8(2):e57135. doi: 10.1371/journal.pone.0057135. PubMed PMID: 23451163; PubMed Central PMCID: PMC3581585.
Kurokawa M, Kim J, Geradts J, Matsuura K, Liu L, Ran X, Xia W, Ribar T J, Henao R, Dewhirst M W, Kim W J, Lucas J E, Wang S, Spector N L, Kornbluth S. A network of substrates of the E3 ubiquitin ligases MDM2 and HUWE1 control apoptosis independently of p53. Science signaling. 2013; 6(274):ra32. doi: 10.1126/scisignal.2003741. PubMed PMID: 23652204; PubMed Central PMCID: PMC3770270.
Lehman H L, Dashner E J, Lucey M, Vermeulen P, Dirix L, Van Laere S, van Golen K L. Modeling and characterization of inflammatory breast cancer emboli grown in vitro. International journal of cancer Journal international du cancer. 2013; 132(10):2283-94. doi: 10.1002/ijc.27928. PubMed PMID: 23129218.
Lerebours F, Vacher S, Andrieu C, Espie M, Marty M, Lidereau R, Bieche I. N F-kappa B genes have a major role in inflammatory breast cancer. BMC cancer. 2008; 8:41. doi: 10.1186/1471-2407-8-41. PubMed PMID: 18248671; PubMed Central PMCID: PMC2267801.
Li C Y, Shan S, Huang Q, Braun R D, Lanzen J, Hu K, Lin P, Dewhirst M W. Initial stages of tumor cell-induced angiogenesis: evaluation via skin window chambers in rodent models. Journal of the National Cancer Institute. 2000; 92(2):143-7. PubMed PMID: 10639516.
Manda G, Nechifor M T, Neagu T-M. Reactive Oxygen Species, Cancer and Anti-Cancer Therapies. Current Chemical Biology. 2009; 3(1):22-46.
Miriyala S, Spasojevic I, Tovmasyan A, Salvemini D, Vujaskovic Z, St Clair D, Batinic-Haberle I. Manganese superoxide dismutase, MnSOD and its mimics. Biochim Biophys Acta. 2012; 1822(5):794-814. doi: 10.1016/j.bbadis.2011.12.002. PubMed PMID: 22198225; PubMed Central PMCID: PMC3304004.
Moeller B J, Batinic-Haberle I, Spasojevic I, Rabbani Z N, Anscher M S, Vujaskovic Z, Dewhirst M W. A manganese porphyrin superoxide dismutase mimetic enhances tumor radioresponsiveness. International journal of radiation oncology, biology, physics. 2005; 63(2):545-52. doi: 10.1016/j.ijrobp.2005.05.026. PubMed PMID: 16168847.
Moeller B J, Cao Y, Li C Y, Dewhirst M W. Radiation activates HIF-1 to regulate vascular radiosensitivity in tumors: role of reoxygenation, free radicals, and stress granules. Cancer cell. 2004; 5(5):429-41. PubMed PMID: 15144951.
Morse M A, Wei J, Hartman Z, Xia W, Ren X R, Lei G, Barry W T, Osada T, Hobeika A C, Peplinski S, Jiang H, Devi G R, Chen W, Spector N, Amalfitano A, Lyerly H K, Clay T M. Synergism from combined immunologic and pharmacologic inhibition of HER2 in vivo. International journal of cancer Journal international du cancer. 2010; 126(12):2893-903. Epub 2009/10/27. doi: 10.1002/ijc.24995. PubMed PMID: 19856307; PubMed Central PMCID: PMC2856803.
Nguyen D M, Sam K, Tsimelzon A, Li X, Wong H, Mohsin S, Clark G M, Hilsenbeck S G, Elledge R M, Allred D C, O'Connell P, Chang J C. Molecular heterogeneity of inflammatory breast cancer: a hyperproliferative phenotype. Clinical cancer research: an official journal of the American Association for Cancer Research. 2006; 12(17):5047-54. Epub 2006/09/05. doi: 10.1158/1078-0432.ccr-05-2248. PubMed PMID: 16951220.
Oldham M, Sakhalkar H, Oliver T, Johnson G A, Dewhirst M. Optical clearing of unsectioned specimens for three-dimensional imaging via optical transmission and emission tomography. Journal of Biomedical Optics. 2008; 13(2):021113-. doi: 10.1117/1.2907968.
Palmer G M, Fontanelle A N, Shan S, Dewhirst M W. High-resolution in vivo imaging of fluorescent proteins using window chamber models. Methods in molecular biology. 2012; 872:31-50. doi: 10.1007/978-1-61779-797-2_3. PubMed PMID: 22700402.
Palmer G M, Fontanelle A N, Shan S, Hanna G, Zhang G, Fraser C L, Dewhirst M W. In vivo optical molecular imaging and analysis in mice using dorsal window chamber models applied to hypoxia, vasculature and fluorescent reporters. Nature protocols. 2011; 6(9):1355-66. doi: 10.1038/nprot.2011.349. PubMed PMID: 21886101; PubMed Central PMCID: PMC3500601.
Raje N, Kumar S, Hideshima T, Ishitsuka K, Yasui H, Chhetri S, Vallet S, Vonescu E, Shiraishi N, Kiziltepe T, Elford H L, Munshi N C, Anderson K C. Didox, a ribonucleotide reductase inhibitor, induces apoptosis and inhibits DNA repair in multiple myeloma cells. British journal of haematology. 2006; 135(1):52-61. doi: 10.1111/j.1365-2141.2006.06261.x. PubMed PMID: 16925573.
Rangwala F, Williams K P, Smith G R, Allensworth J, Thomas Z, Lyerly H K, Diehl A M, Morse M A, Devi G R. Differential effects of arsenic trioxide on chemosensitization in human hepatic tumor and stellate cell lines. BMC cancer. 2012; 12(1):402. doi: 10.1186/1471-2407-12-402. PubMed PMID: 22963400.
Robertson F, Cristofanilli M. New Therapeutic Targets in Inflammatory Breast Cancer. Curr Breast Cancer Rep. 2012; 4(4):264-70. doi: 10.1007/s12609-012-0087-3.
Robertson F M, Bondy M, Yang W, Yamauchi H, Wiggins S, Kamrudin S, Krishnamurthy S, Le-Petross H, Bidaut L, Player A N, Barsky S H, Woodward W A, Buchholz T, Lucci A, Ueno N T, Cristofanilli M. Inflammatory breast cancer: the disease, the biology, the treatment. C A: a cancer journal for clinicians. 2010; 60(6):351-75. doi: 10.3322/caac.20082. PubMed PMID: 20959401.
Rossi R, Dalle-Donne I, Milzani A, Giustarini D. Oxidized forms of glutathione in peripheral blood as biomarkers of oxidative stress. Clinical chemistry. 2006; 52(7):1406-14. Epub 2006/05/13. doi: 10.1373/clinchem.2006.067793. PubMed PMID: 16690733.
Rubens R D, Kaye S B, Soukop M, Williams C J, Brampton M H, Harris A L. Phase II trial of didox in advanced breast cancer. Cancer Research Campaign Phase I/II Clinical Trials Committee. Br J Cancer. 1991; 64(6):1187-8. PubMed PMID: 1764386; PubMed Central PMCID: PMC1977835.
Saigal K, Hurley J, Takita C, Reis I M, Zhao W, Rodgers S E, Wright J L. Risk factors for locoregional failure in patients with inflammatory breast cancer treated with trimodality therapy. Clinical breast cancer. 2013; 13(5):335-43. doi: 10.1016/j.clbc.2013.04.002. PubMed PMID: 23850216.
Sarkisyan G, Cahalan S M, Gonzalez-Cabrera P J, Leaf N B, Rosen H. Real-time differential labeling of blood, interstitium, and lymphatic and single-field analysis of vasculature dynamics in vivo. American journal of physiology Cell physiology. 2012; 302(10):C1460-8. doi: 10.1152/ajpce11.00382.2011. PubMed PMID: 22357735.
Schito L, Rey S, Tafani M, Zhang H, Wong C C, Russo A, Russo M A, Semenza G L. Hypoxia-inducible factor 1-dependent expression of platelet-derived growth factor B promotes lymphatic metastasis of hypoxic breast cancer cells. Proceedings of the National Academy of Sciences of the United States of America. 2012; 109(40):E2707-16. doi: 10.1073/pnas.1214019109. PubMed PMID: 23012449; PubMed Central PMCID: PMC3479586.
Sekhon H S, London C A, Sekhon M, Iversen P L, Devi G R. c-MYC antisense phosphosphorodiamidate morpholino oligomer inhibits lung metastasis in a murine tumor model. Lung cancer. 2008; 60(3):347-54. doi: 10.1016/j.lungcan.2007.10.028. PubMed PMID: 18096271.
Semenza G L. Oxygen sensing, homeostasis, and disease. The New England journal of medicine. 2011; 365(6):537-47. doi: 10.1056/NEJMra1011165. PubMed PMID: 21830968.
Shah K N, Mehta K R, Peterson D, Evangelista M, Livesey J C, Faridi J S. AKT-induced tamoxifen resistance is overturned by RRM2 inhibition. Molecular cancer research: MCR. 2014; 12(3):394-407. doi: 10.1158/1541-7786.MCR-13-0219. PubMed PMID: 24362250.
Shan S, Robson N D, Cao Y, Qiao T, Li C Y, Kontos C D, Garcia-Blanco M, Dewhirst M W. Responses of vascular endothelial cells to angiogenic signaling are important for tumor cell survival. FASEB journal: official publication of the Federation of American Societies for Experimental Biology. 2004; 18(2):326-8. doi: 10.1096/fj.03-0765fje. PubMed PMID: 14688196.
Spitz D R, Oberley L W. An assay for superoxide dismutase activity in mammalian tissue homogenates. Analytical biochemistry. 1989; 179(1):8-18. Epub 1989/05/15. PubMed PMID: 2547324.
Thomas A, Bowsher J, Roper J, Oliver T, Dewhirst M, Oldham M. A comprehensive method for optical-emission computed tomography. Physics in Medicine and Biology. 2010; 55(14):3947-57. doi: 10.1088/0031-9155/55/14/001.
Thomas Z I, Gibson W, Sexton J Z, Aird K M, Ingram S M, Aldrich A, Lyerly H K, Devi G R, Williams K P. Targeting GUI expression in human inflammatory breast cancer cells enhances apoptosis and attenuates migration. Br J Cancer. 2011; 104(10):1575-86. Epub 2011/04/21. doi: 10.1038/bjc.2011.133. PubMed PMID: 21505458; PubMed Central PMCID: PMC3101910.
Toyokuni S, Okamoto K, Yodoi J, Hiai H. Persistent oxidative stress in cancer. FEBS letters. 1995; 358(1):1-3. PubMed PMID: 7821417.
Trachootham D, Alexandre J, Huang P. Targeting cancer cells by ROS-mediated mechanisms: a radical therapeutic approach? Nature reviews Drug discovery. 2009; 8(7):579-91. doi: 10.1038/nrd2803. PubMed PMID: 19478820.
Truman L A, Bentley K L, Smith E C, Massaro S A, Gonzalez D G, Haberman A M, Hill M, Jones D, Min W, Krause D S, Ruddle N H. ProxTom lymphatic vessel reporter mice reveal Prox1 expression in the adrenal medulla, megakaryocytes, and platelets. The American journal of pathology. 2012; 180(4):1715-25. doi: 10.1016/j.ajpath.2011.12.026. PubMed PMID: 22310467; PubMed Central PMCID: PMC3349900.
Van Laere S J, Van der Auwera I, Van den Eynden G G, Elst H J, Weyler J, Harris A L, van Dam P, Van Marck E A, Vermeulen P B, Dirix L Y. Nuclear factor-kappaB signature of inflammatory breast cancer by cDNA microarray validated by quantitative real-time reverse transcription-PCR, immunohistochemistry, and nuclear factor-kappaB DNA-binding. Clinical cancer research: an official journal of the American Association for Cancer Research. 2006; 12(11 Pt 1):3249-56. PubMed PMID: 16740744.
Van Laere S J, Van der Auwera I, Van den Eynden G G, van Dam P, Van Marck E A, Vermeulen P B, Dirix L Y. NF-kappaB activation in inflammatory breast cancer is associated with oestrogen receptor downregulation, secondary to EGFR and/or ErbB2 overexpression and MAPK hyperactivation. Br J Cancer. 2007; 97(5):659-69. Epub 2007/08/19. doi: 6603906 [pii]10.1038/sj.bjc.6603906 [doi]. PubMed PMID: 17700572; PubMed Central PMCID: PMC2360371.
Vermeulen P B, van Golen K L, Dirix L Y. Angiogenesis, lymphangiogenesis, growth pattern, and tumor emboli in inflammatory breast cancer: a review of the current knowledge. Cancer. 2010; 116(11 Suppl):2748-54. doi: 10.1002/cncr.25169. PubMed PMID: 20503405.
Viola R J, Provenzale J M, Li F, Li C Y, Yuan H, Tashjian J, Dewhirst M W. In vivo bioluminescence imaging monitoring of hypoxia-inducible factor 1alpha, a promoter that protects cells, in response to chemotherapy. AJR American journal of roentgenology. 2008; 191(6):1779-84. doi: 10.2214/AJR.07.4060. PubMed PMID: 19020250.
Williams K P, Allensworth J L, Ingram S M, Smith G R, Aldrich A J, Sexton J Z, Devi G R. Quantitative high-throughput efficacy profiling of approved oncology drugs in inflammatory breast cancer models of acquired drug resistance and re-sensitization. Cancer letters. 2013; 337(1):77-89. doi: 10.1016/j.canlet.2013.05.017. PubMed PMID: 23689139; PubMed Central PMCID: PMC3763836.
Yang D, Zhao Y, Li A Y, Wang S, Wang G, Sun Y. Smac-mimetic compound SM-164 induces radiosensitization in breast cancer cells through activation of caspases and induction of apoptosis. Breast cancer research and treatment. 2012; 133(1):189-99. doi: 10.1007/s10549-011-1752-3. PubMed PMID: 21901386.
Zagar T M, Oleson J R, Vujaskovic Z, Dewhirst M W, Craciunescu O I, Blackwell K L, Prosnitz L R, Jones E L. Hyperthermia combined with radiation therapy for superficial breast cancer and chest wall recurrence: a review of the randomised data. International journal of hyperthermia: the official journal of European Society for Hyperthermic Oncology, North American Hyperthermia Group. 2010; 26(7):612-7. doi: 10.3109/02656736.2010.487194. PubMed PMID: 20849256; PubMed Central PMCID: PMC2956421.
Zar J. Biostatistical Analysis2010.

### Example 5: High Content Analysis and Targeting Cancer-Specific Pathway in Three Dimensional Inflammatory Breast Cancer Tumor Spheroids

Patients with inflammatory breast cancer (IBC) have a very poor prognosis. Their tumors are not responsive to chemotherapy. Previous studies have suggested that metastasis in IBC is driven by the formation of tumor spheroids, which are specialized multicellular structures (also referred to as tumor emboli in the lymphatic system) that collectively migrate and spread to distant organs. It is therefore critical to understand how cells in the tumor spheroids evade the process of programmed cell death to develop novel anti-cancer drugs to target these mechanisms.

The objectives of this study were to: 1) define characteristics of tumor cell spheroids that enable them to resist therapy, 2) develop a novel 3D in vitro model of spheroid/emboli formation to identify therapeutic strategies to treat them. This study focused on the molecular and phenotypic characteristics that promote their survival by evaluating expression of anti-cell death proteins in breast tumor tissue microarrays, developing a high content assay, and then applying this assay to identify compounds that induce cell death and inhibit tumor spheroid formation. Immunohistochemistry analysis showed that the IBC tumor spheroids express high levels of a potent anti-cell death protein, X-linked Inhibitor of apoptosis protein (XIAP), and the nuclear transcription factor, NFκB, a pro-cell survival protein.

The 3D IBC tumor spheroid-high content assay developed was used to identify novel XIAP and NFκB inhibitors that target tumor spheroids. Translation to clinical applications will require further testing of these identified compounds in preclinical drug development models. The 3D tumor spheroid-high content assay can be used to simulate the tumor microenvironment, and facilitate high-throughput drug screening.

3D Cell Health IBC Multi Parametric System (3D CHIMPS) a High Content Analysis and Targeting Cancer-Specific Pathway in Three Dimensional Breast Cancer Spheroids

The molecular basis of how tumor spheroids evade apoptosis is poorly understood. Cancer cells gain mechanisms to avoid apoptosis. XIAP, X-linked inhibitor of apoptosis protein is the most potent anti-apoptotic/anti cell death protein known to date. XIAP can activate a nuclear transcription factor (NFκB) which could mediate tumor spheroid survival. Up until now, there has been a lack of quantitative in vitro tumor spheroid models. Tumor spheroids/tumor emboli are multicellular structures. We describe in this example and disclosure an in vitro cell-based assay that can quantitatively measure the tumor spheroid phenotype and viability for drug discovery and improved therapies.

In this example, we demonstrate 1) IBC tumor emboli expresss anti-cell death proteins, XIAP and NFκB in tumor spheroids in vivo, 2) our new high content assay for simultaneous imaging and quantitative measurement of IBC tumor spheroid morphology and viability, and 3) application of the tumor spheroid-high content model to evaluate candidate compounds that target XIAP and NFκB for their ability to inhibit tumor spheroid formation and viability.

Immunohistochemical Analysis of Anti-Apoptotic Proteins XIAP and NFκB in Breast Cancer Samples

Tissue microarrays consisting of de-identified patient breast tumors were analyzed for XIAP and NFκB expression using immunohistochemistry. The steps are outlined in FIG. 12 using a primary antibody to target XIAP or NFκB protein, adding a secondary antibody linked to a detection agent (e.g. horse radish peroxidase (HRP)), adding DAB substrate for enzyme HRP, washing and adding slide coverslips, mounting and scoring using microscopy. Ad shown in the representative figures in FIG. 12, there was intense cytoplasmic staining of XIAP and both cytoplasmic and nuclear staining of NFκB in invasive breast tumors including all IBC tested. Negative staining was generally observed in samples of benign and non-cancerous breast epithelium. Cluster of tumor cells (spheroid-like) were evident only in IBC tumor samples and these stained positive for both XIAP and NFκB (yellow arrows, FIG. 12).

Development of the High Content Tumor Spheroid Assay

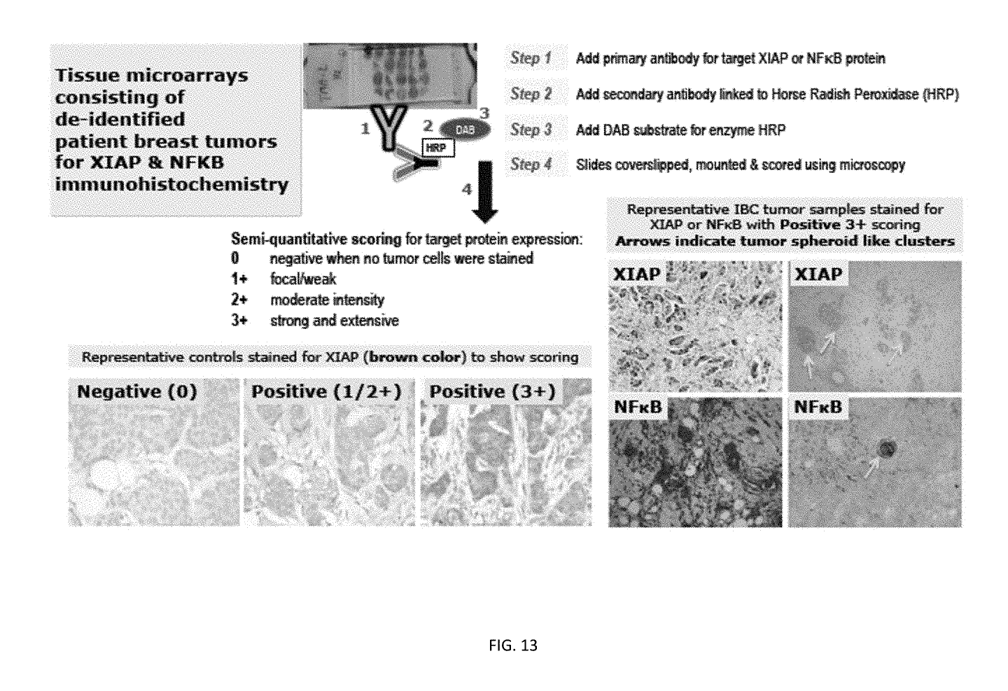

SUM149 human cell line is considered a true IBC-like cell model derived from a patient tumor. SUM149 cells express XIAP and NFκB, and therefore SUM149 cells were used to develop the high content tumor spheroid assay as depicted in FIG. 13. As outlined in FIG. 13, appropriate numbers of SUM149 cells were seeded in ultra-low attachment multi-well plates (6, 12, or 96 well). 3 dimensional (3D) tumor spheroids were observed daily by phase contrast microscopy. By day 2, tumor spheroids formed. Cells were then stained (Hoechst, YOYO-1 and MitoTracker Red) and high content image analysis was performed. Imaging of multiple spheroids after masking (orange) to exclude small spheroids (<50 μm diameter). Uniform staining for Hoechst (nuclear) and MitrTracker Red (mitochondria) with little to no YOYO-1 (staining only in cells dying) indicates viable tumor spheroid formation. FIG. 13 demonstrates formation of the 3D IBC tumor spheroids in the high content, high-throughput assay system developed.

High Content 3D Tumor Spheroid Assay to Evaluate Efficacy of Known XIAP and NFκB Inhibitors

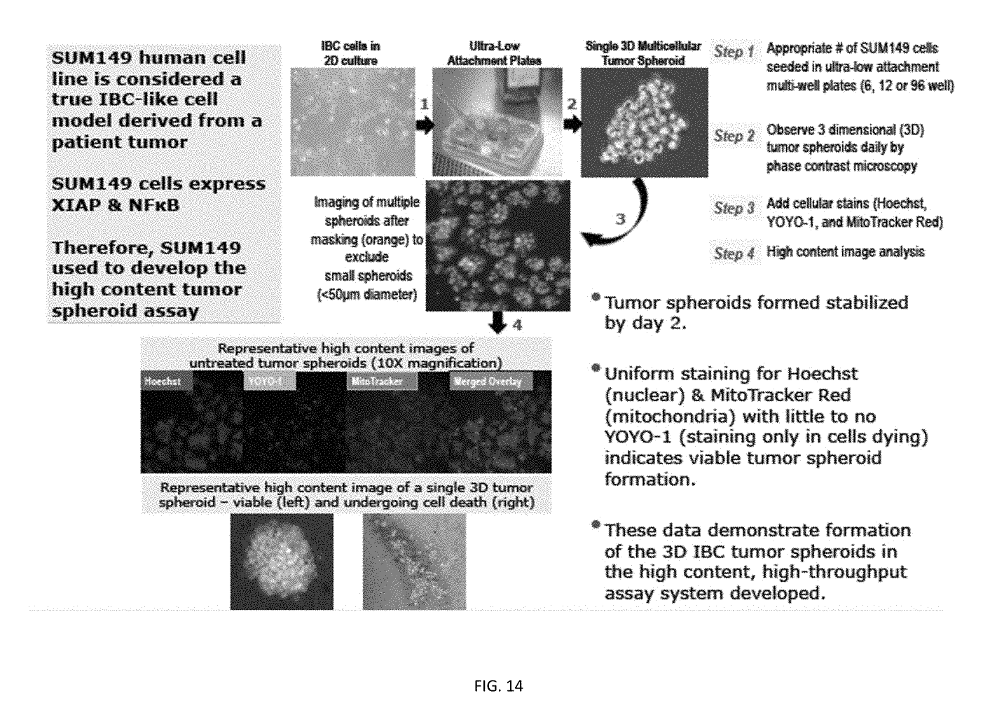

As depicted in FIG. 14, the high content 3D tumor spheroid assay of the present invention was used to evaluate efficacy of known XIAP and NFκB inhibitors. Treatments with selected compounds specifically targeting XIAP and NFκB or non-targeted compounds (doses bases on previous efficacy studies in regular 2D SUM149 cultures). Treatments carried out at the time of cell seeding in ultra-low attachment plates to assess tumor spheroid formation. As demonstrated in FIG. 14, untreated control have viable tumor spheroids. A non-targeting control compound (does not target XIAP or NFκB) was similar to untreated. Surprisingly, XIAP inhibitors tested did not inhibit spheroid formation but did seem to lead to cell death. However, cell death (yellow arrows) observed in spheroid core (XIAP Inhibitor #1) outer edges (XIAP Inhibitor #2). NFκB inhibitors #1 and #2 tested inhibit spheroid formation, causing massive cell death.

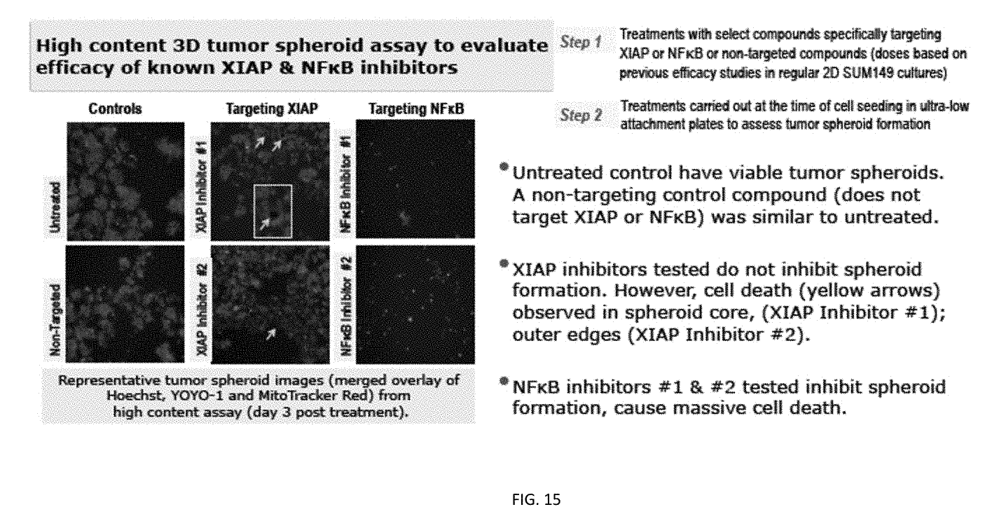

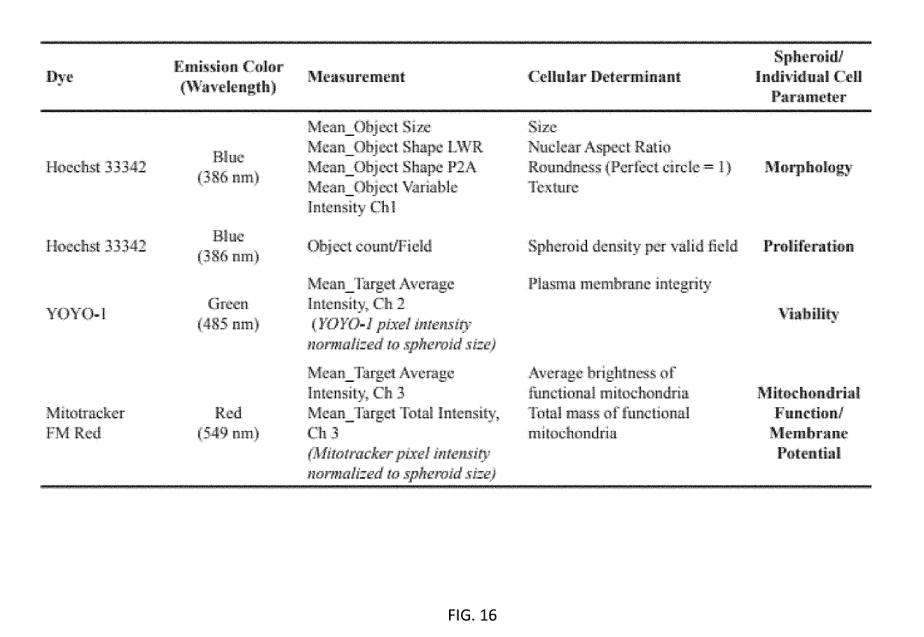

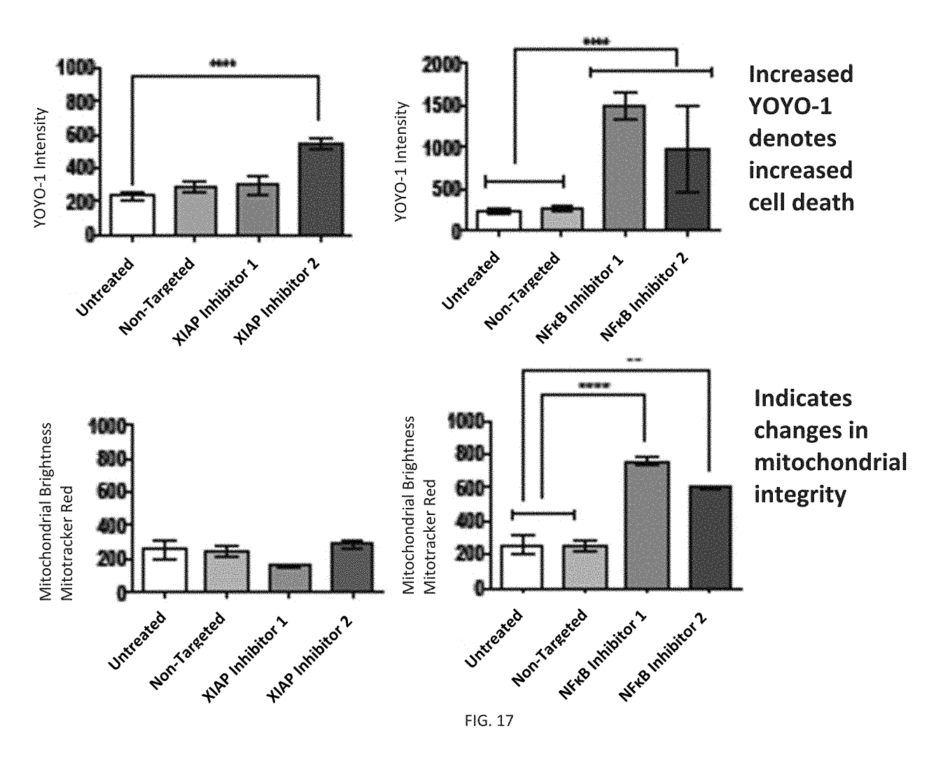

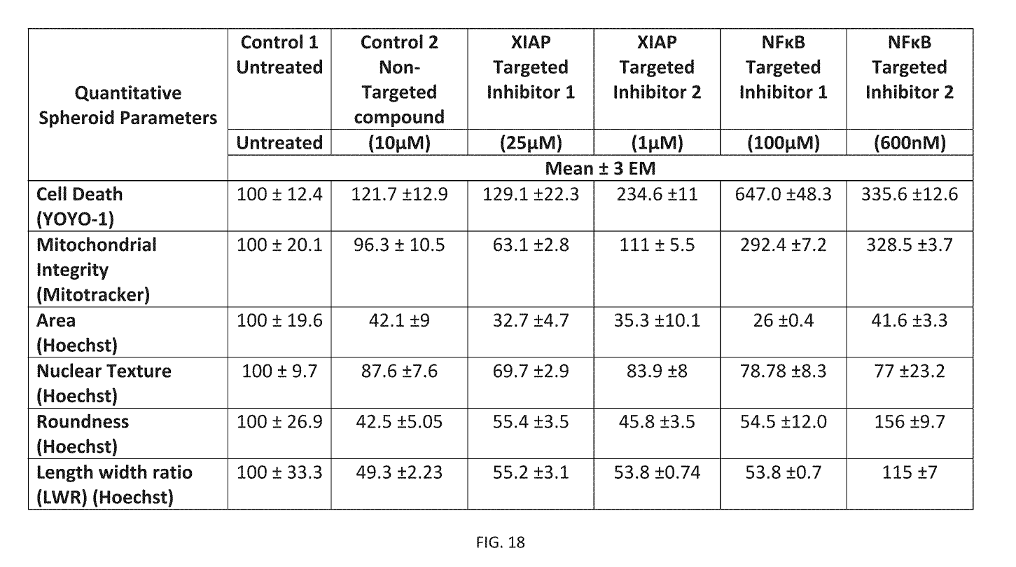

High content imaging data from untreated and treated tumor spheroids were analyzed simultaneously for quantitative cellular parameters as shown in the table in FIG. 15. The image data was statistically analyzed using quantitative measurements by the 3 channel cell health profiling protocol in FIG. 15 from 150-175 fields of view per well, with 2-3 wells per treatment. One-way ANOVA, Tukey Multiple Comparison Test was used and the results are shown in the table of FIG. 16 and FIG. 17 are graphs showing the statistical analysis of the image data, FIG. 18 shows the summary of results. Data was considered significant if p<0.05. Results demonstrate that IBC express high levels of anti-apoptotic proteins and that targeting these proteins can inhibit tumor spheroids.

This is the first time it has been demonstrated that XIAP is overexpressed in IBC patient tumors and tumor spheroids, and thus van be used for identifying a target for drug development. A high content, high-throughput in vitro tumor spheroid assay developed can be used for simultaneous imaging, morphometric and viability analysis.

Any patents or publications mentioned in this specification are indicative of the levels of those skilled in the art to which the invention pertains. These patents and publications are herein incorporated by reference to the same extent as if each individual publication was specifically and individually indicated to be incorporated by reference. In case of conflict, the present specification, including definitions, will control.

One skilled in the art will readily appreciate that the present invention is well adapted to carry out the objects and obtain the ends and advantages mentioned, as well as those inherent therein. The present disclosure described herein are presently representative of preferred embodiments, are exemplary, and are not intended as limitations on the scope of the invention. Changes therein and other uses will occur to those skilled in the art which are encompassed within the spirit of the invention as defined by the scope of the claims.

The present invention is not intended to be limited to the examples, but encompasses all such modifications and variations as come within the scope of the appended claims.

1. A high-content, high throughput multiparametric assay method for analyzing 3-D Inflammatory Breast Cancer (IBC) tumor emboli comprising:
a) generating 3D IBC spheroid array in in vitro culture by
(i) plating IBC cells on ultra low attachment plates;
(ii) culturing the IBC cells for at least 2 days to form 3-dimensional tumor emboli;
b) simultaneously assaying the tumor emboli cell array for at least two cell health parameters selected from the group consisting of cell viability, cell number, nuclear shape, nuclear size, nuclear texture, cell proliferation, and mitochondrial function (mitochondrial membrane potential), wherein measuring comprises simultaneous cell staining, cell imaging, or a combination thereof, and
c) analyzing the at least two parameters of step (b).

2. The method of claim 1, wherein the wherein step (b) comprises
(i) incubating the 3D IBC spheroid array with at least one marker for a cell health parameter selected from the group consisting of Hoechst, YOYO-1 and Mitotracker™ Red.

3. The method of claim 1, wherein step (c) comprises quantitating signal from the detection agent.

4. The method of claim 1, wherein the step (a) comprises plating and culturing the IBC cells in chemically defined medium.

5. The method of claim 1, wherein the IBC cells are selected from the group consisting of SUM149, rSUM149 and SUM190 IBC cells.

6. The method of claim 5, wherein the IBC cells are SUM149 cell line.

7. The method of claim 1, wherein the IBC cells are derived from an IBC tissue sample from a patient.

8. A method for high throughput identification of an agent for IBC tumor emboli cell growth inhibition, the method comprising:
a) generating a 3D IBC spheroid array in an in vitro culture by
(i) plating IBC cells on ultra low attachment plates; and
(ii) culturing the IBC cells for at least 2 days to form 3-dimensional tumor emboli; and
b) providing at least one agent to be tested;
c) contacting the IBC tumor emboli array with the agent; and
d) detecting at least one signal indicating IBC tumor emboli cells growth inhibition as compared to a non-contacted control.

9. The method of claim 8, wherein the at least one signal indicating IBC tumor emboli cell inhibition is selected from the group consisting of (i) a reduction in tumor emboli cell number, (ii) an increase in cell death, (iii) inhibition of spheroid formation, (iv) reduction of mitochondrial integrity, and (v) a combination thereof.

10. The method of claim 9, wherein the signal indicating IBC tumor emboli cell growth inhibition is an increase in cell death and wherein step (d) comprises incubating the 3D IBC spheroid array with at least one marker of cell death, wherein the an increase in the marker is an indicator of cell death.

11. The method of claim 10, wherein the marker of cell death is a fluorescent dye and the method comprises measuring the uptake of a fluorescent dye using high resolution imaging, wherein uptake of the fluorescent dye indicates cell death.

12. The method of claim 11, wherein the marker of cell death is YOYO-1.

13. The method of claim 9, wherein the signal indicating IBC tumor emboli cell growth inhibition in reduction of mitochondrial integrity and wherein step (d) comprises incubating the 3D IBC spheroid array with a marker of mitochondrial integrity.

14. The method of claim 13, wherein the marker of mitochondrial integrity is Mitotracker™ Red.

15. The method of claim 8, wherein the at least one signal is at least three signals selected from the group consisting of (i) a reduction in tumor emboli cell number, (ii) an increase in cell death, (iii) inhibition of spheroid formation, (iv) reduction of mitochondrial integrity, and (v) a combination thereof

16. The method of claim 8, wherein the agent to be tested is an inhibitor of XIAP or NFκB.

17. The method of claim 16, wherein step (d) comprises incubating the 3D IBC spheroid array with Hoechst, YOYO-1, and Mitotracker Red and measuring Hoechst, YOYO-1, and Mitotracker Red staining as an indication of cell morphology, cell viability, and mitochondrial function, respectively.

18. The method of claim 8, wherein step (d) comprises incubating the 3D IBC spheroid array with at least one fluorescent marker and using a microscope to image the array, wherein changes in fluorescence in the images compared to the non-contacted control incubated with the at least one fluorescent marker indicate IBC tumor emboli cell growth inhibition.

19. The method of claim 18, wherein the images of the contacted wells are quantitated as compared to non-contacted control wells.

20. An in vitro 3-dimensional Inflammatory Breast Cancer Tumor Emboli high content throughput assay comprising:
a) an array of 3-dimensional IBC tumor emboli within multiple culture wells generated in vitro by
(i) plating IBC cells in wells of an ultra low attachment plate;
(ii) culturing the IBC cells for at least 2 days to form 3-dimensional IBC tumor emboli;
b) at least one detection agent able to produce a detectable signal, wherein the detectable signal is selected from the group consisting of (i) a reduction in tumor emboli cell number, (ii) an increase in cell death, (iii) inhibition of spheroid formation, (iv) reduction of mitochondrial integrity, and (v) a combination thereof;
c) a microscope for imaging the detectable signal, and
d) means for quantitatively analyzing the images.
